(19) United States
(12) Patent Application Publication
(10) Pub. No.: US 2005/0287121 A1
(43) Pub. Date: Dec. 29, 2005

(54) Methods, compositions and compound assays for inhibiting amyloid-beta protein production

(76) Inventors: Pascal Gerard Merchiers, Tielen (BE); Marcel Hoffmann, Uithoorn (NL); Koenraad Frederik Florentina Spittaels, Puurs (BE); Wendy Laenen, Lier (BE)

Correspondence Address: SYNNESTVEDT & LECHNER, LLP, 2600 ARAMARK TOWER, 1101 MARKET STREET, PHILADELPHIA, PA, 191072950, US

(21) Appl. No.: 11/127,622

(22) Filed: May 12, 2005

Related U.S. Application Data
(60) Provisional application No. 60/570,352, filed on May 12, 2004.
(60) Provisional application No. 60/603,948, filed on Aug. 24, 2004.

(51) Int. Cl. A61K 48/00; G01N 33/53

(52) U.S. Cl. 424/93.2; 435/7.1

(57) ABSTRACT

A method for identifying compounds that inhibit aberrant amyloid-beta precursor protein processing in cells, comprising contacting a test compound with a LIGASE polypeptide, or fragment thereof, and measuring a compound-LIGASE property related to the production of amyloid-beta peptide. Cellular assays of the method measure amyloid beta peptide levels. Therapeutic methods, and pharmaceutical compositions including effective amyloid-beta precursor processing-inhibiting amounts of LIGASE DNA expression agents or LIGASE agonists, are useful for treating conditions involving cognitive impairment such as Alzheimers Disease.

### CROSS-REFERENCE TO RELATED APPLICATIONS

This application claims priority to U.S. Provisional Application No. 60/570,352, filed May 12, 2004, and U.S. Provisional Application No. 60/603,948, filed Aug. 24, 2004, the disclosures of which are incorporated herein by reference.

### FIELD OF THE INVENTION

This invention relates to the field of mammalian neuronal cell disorders, and in particular, to methods for identifying effective compounds, and therapies and compositions using such compounds, useful for the prevention and treatment of diseases associated with progressive loss of intellectual capacities in humans.

The neurological disorder that is most widely known for its progressive loss of intellectual capacities is Alzheimer's disease (AD). Worldwide, about 20 million people suffer from Alzheimer's disease. AD is clinically characterized by the initial loss of memory, followed by disorientation, impairment of judgment and reasoning, which is commonly referred to as cognitive impairment, and ultimately by full dementia. AD patients finally lapse into a severely debilitated, immobile state between four and twelve years after onset of the disease.

The key pathological evidence for AD is the presence of extracellular amyloid plaques and intracellular tau tangles in the brain, which are associated with neuronal degeneration (Ritchie and Lovestone (2002)). The extracellular amyloid plaques are believed to result from an increase in the insoluble amyloid beta peptide 1-42 produced by the metabolism of amyloid-beta precursor protein (APP). Following secretion, these amyloid beta 1-42 peptides form amyloid fibrils more readily than the amyloid beta 1-40 peptides, which are predominantly produced in healthy people. It appears that the amyloid beta peptide is on top of the neurotoxic cascade: experiments show that amyloid beta fibrils, when injected into the brains of P301L tau transgenic mice, enhance the formation of neurofibrillary tangles (Gotz et al. (2001)). In fact, a variety of amyloid beta peptides have been identified as amyloid beta peptides 1-42, 1-40, 1-39, 1-38, 1-37, which can be found in plaques and are often seen in cerebral spinal fluid.

The amyloid beta peptides are generated (or processed) from the membrane anchored APP, after cleavage by beta secretase and gamma secretase at position 1 and 40 or 42, respectively (FIG. 1A)(Annaert and De Strooper (2002)). In addition, high activity of beta secretase results in a shift of the cleavage at position 1 to position 11. Cleavage of amyloid-beta precursor protein by alpha secretase activity at position 17 and gamma secretase activity at 40 or 42 generates the non-pathological p3 peptide. Beta secretase was identified as the membrane anchored aspartyl protease BACE, while gamma secretase is a protein complex comprising presenilin 1 (PS1) or presenilin 2 (PS2), nicastrin, Anterior Pharynx Defective 1 (APH1) and Presenilin Enhancer 2 (PEN2). Of these proteins, the presenilins are widely thought to constitute the catalytic activity of the gamma secretase, while the other components play a role in the maturation and localization of the complex. The identity of the alpha secretase is still illustrious, although some results point towards the proteases ADAM 10 and TACE, which could have redundant functions.

A small fraction of AD cases (mostly early onset AD) are caused by autosomal dominant mutations in the genes encoding presenilin 1 and 2 (PS1; PS2) and the amyloid-beta precursor protein (APP), and it has been shown that mutations in APP, PS1 and PS2 alter the metabolism of amyloid-beta precursor protein leading to such increased levels of amyloid beta 1-42 produced in the brain. Although no mutations in PS1, PS2 and amyloid-beta precursor protein have been identified in late onset AD patients, the pathological characteristics are highly similar to the early onset AD patients. These increased levels of amyloid beta peptide could originate progressively with age from disturbed amyloid-beta precursor protein processing (e.g. high cholesterol levels enhance amyloid beta peptide production) or from decreased amyloid beta peptide catabolism. Therefore, it is generally accepted that AD in late onset AD patients is also caused by aberrant increased amyloid peptide levels in the brains. The level of these amyloid beta peptides, and more particularly amyloid-beta peptide 1-42, is increased in Alzheimer patients compared to the levels of these peptides in healthy persons. Thus, reducing the levels of these amyloid beta peptides is likely to be beneficial for patients with cognitive impairment.

### REFERENCES

Annaert, W. and B. De Strooper (2002). “A cell biological perspective on Alzheimer's disease.” Annu Rev Cell Dev Biol 18: 25-51.
Gotz, J., F. Chen, et al. (2001). “Formation of neurofibrillary tangles in P3011 tau transgenic mice induced by Abeta 42 fibrils.” Science 293(5534): 1491-5.
Lipinski, C. A., Lombardo, F., Dominy, B. W., and Feeney, P. J. Adv. Drug. Deliv. Rev., 23, 3-25, 1997
Ritchie, K. and S. Lovestone (2002). “The dementias.” Lancet 360(9347): 1759-66.

### Reported Developments

The major current AD therapies are limited to delaying progressive memory loss by inhibiting the acetylcholinesterase enzyme, which increases acetylcholine neurotransmitter levels, which fall because the cholinergic neurons are the first neurons to degenerate during AD. This therapy does not halt the progression of the disease.

Therapies aimed at decreasing the levels of amyloid beta peptides in the brain, are increasingly being investigated and focus on the perturbed amyloid-beta precursor protein processing involving the beta- or gamma secretase enzymes.

The present invention is based on the discovery that certain known polypeptides are factors in the up-regulation and/or induction of amyloid beta precursor processing in neuronal cells, and that the inhibition of the function of such polypeptides are effective in reducing levels of amyloid beta peptides.

### SUMMARY OF THE INVENTION

The present invention relates to the relationship between the function of the type 1 transmembrane protein, RNF128 (“LIGASE”) and amyloid-beta precursor protein processing in mammalian cells.

One aspect of the present invention is a method for identifying a compound that inhibits the processing of amyloid-beta precursor protein in a mammalian cell, comprising
(a) contacting a compound with a LIGASE polypeptide; and
(b) measuring a compound-polypeptide property related to the production of amyloid-beta protein.

Aspects of the present method include the in vitro assay of compounds using polypeptide domains comprising an amino acid sequence of SEQ ID NO: 2, 3, or 5, and cellular assays wherein LIGASE modulation is followed by observing indicators of efficacy, including amyloid beta peptide levels.

Another aspect of the invention is a method of treatment or prevention of a condition involving cognitive impairment, or a susceptibility to the condition, in a subject suffering or susceptible thereto, by administering a pharmaceutical composition comprising an effective amyloid-beta precursor processing-modulating amount of a LIGASE agonist.

A further aspect of the present invention is a pharmaceutical composition for use in said method wherein said LIGASE agonist comprises a polynucleotide sequence encoding a polypeptide comprising an amino acid sequence of SEQ ID NO: 1.

Another further aspect of the present invention is a pharmaceutical composition comprising a therapeutically effective amyloid-beta precursor processing-modulating amount of a LIGASE agonist or its pharmaceutically acceptable salt, hydrate, solvate, or prodrug thereof in admixture with a pharmaceutically acceptable carrier. The present polynucleotides and LIGASE agonist compounds are also useful for the manufacturing of a medicament for the treatment of Alzheimer's disease.

### BRIEF DESCRIPTION OF THE DRAWINGS

FIG. 1A. APP processing: The membrane anchored amyloid precursor protein (APP) is processed by two pathways: the amyloidogenic and non-amyloidogenic pathway.

In the latter pathway, APP is cleaved first by alpha secretase and then by gamma secretase, yielding the p3 peptides (17-40 or 17-42). The amyloidogenic pathway generates the pathogenic amyloid beta peptides (A beta) after cleavage by beta- and gamma-secretase respectively. The numbers depicted are the positions of the amino acids comprising the A beta sequences.

FIG. 2. Evaluation of the APP processing assay: Positive (PS1G384L; PS1L392V and BACE1) and negative (eGFP, LacZ and empty) control viruses are infected in Hek293APPwt at random MOI, mimicking a screening. A and B: Transduction is performed respectively with 1 and 0.2 μl of virus and amyloid beta 1-42 levels are performed. Data are represented as relative light units and correlate to pM of amyloid beta 1-42.

FIG. 3. Evaluation of the APP processing assay: Positive (PS1G384L; PS1L392V and BACE1) and negative (eGFP, LacZ and empty) control viruses are infected in SH-SYSY APPwt at random MOI, mimicking a screening. Transduction is performed with 1 μl of virus and amyloid beta 1-42 levels (A) or amyloid beta x-42 levels (B) are determined. Data are represented as relative light units and correlate to pM of amyloid beta 1-42 and x-42.

FIG. 4. Positive (PS1G384L and BACE1) and negative (eGFP, LacZ and empty) control viruses are infected in Hek293APPwt at random MOI. Transduction is performed respectively with 0.2 μl of virus and amyloid beta 1-42 levels are determined. Data are represented as single relative light units data points. The average and standard deviation of all negative controls is calculated and the cut off is determined using the AVERAGE+(3*STDEV) formula. The cut off is depicted as a line. All positive controls are clearly positioned above the cut-off.

FIG. 5. Hek293 APPwt cells are infected with either empty adenovirus or adenovirus expressing LIGASE. The amyloid beta 1-42 (A), x-42 (B), 1-40 (C) and 1-x (D) levels are measured with the corresponding amyloid beta ELISA.

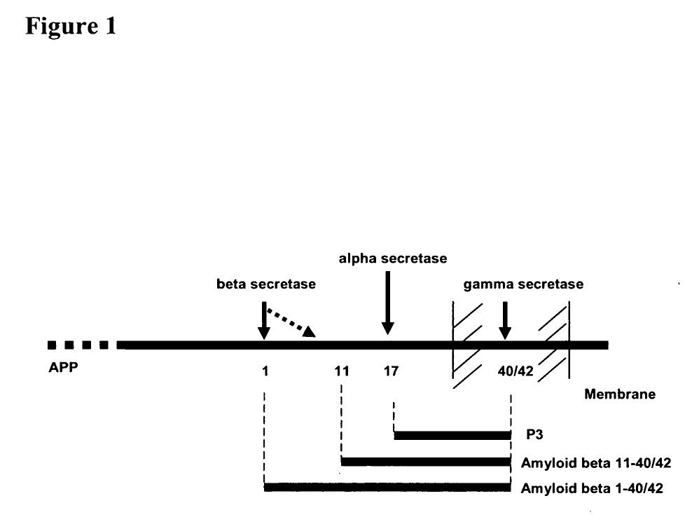

### DETAILED DESCRIPTION

The following terms are intended to have the meanings presented therewith below and are useful in understanding the description of and intended scope of the present invention.

Definitions:

The term “agonist” refers to a ligand that activates the intracellular response of the receptor to which the agonist binds.

The term “amyloid beta peptide” means amyloid beta peptides processed from the amyloid beta precursor protein (APP). The most common peptides include amyloid beta peptides 1-40, 1-42, 11-40 and 11-42. Other species less prevalent amyloid beta peptides are described as x-42, whereby x ranges from 2-10 and 12-17, and 1-y whereby y ranges from 24-39 and 41. For descriptive and technical purposes hereinbelow, “x” has a value of 2-17, and “y” has a value of 24 to 41.

The term “antagonist” means a moiety that bind competitively to the receptor at the same site as the agonists but which do not activate the intracellular response initiated by the active form of the receptor, and can thereby inhibit the intracellular responses by agonists. Antagonists do not diminish the baseline intracellular response in the absence of an agonist or partial agonist.

The term “carrier” means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as TWEEN™, polyethylene glycol (PEG), and PLURONICS™.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

The term “constitutive receptor activation” means stabilization of a receptor in the active state by means other than binding of the receptor with its endogenous ligand or a chemical equivalent thereof.

The term “contact” or “contacting” means bringing at least two moieties together, whether in an in vitro system or an in vivo system.

The term “condition” or “disease” means the overt presentation of symptoms (i.e., illness) or the manifestation of abnormal clinical indicators (e.g., biochemical indicators), resulting from defects in one amyloid beta protein precursor processing. Alternatively, the term “disease” refers to a genetic or environmental risk of or propensity for developing such symptoms or abnormal clinical indicators.

The term “endogenous” shall mean a material that a mammal naturally produces. Endogenous in reference to, for example and not limitation, the term “receptor” shall mean that which is naturally produced by a mammal (for example, and not limitation, a human) or a virus. In contrast, the term non-endogenous in this context shall mean that which is not naturally produced by a mammal (for example, and not limitation, a human) or a virus. For example, and not limitation, a receptor which is not constitutively active in its endogenous form, but when manipulated becomes constitutively active, is most preferably referred to herein as a “non-endogenous, constitutively activated receptor.” Both terms can be utilized to describe both “in vivo” and “in vitro” systems. For example, and not a limitation, in a screening approach, the endogenous or non-endogenous receptor may be in reference to an in vitro screening system. As a further example and not limitation, where the genome of a mammal has been manipulated to include a non-endogenous constitutively activated receptor, screening of a candidate compound by means of an in vivo system is viable.

The term “expression” comprises both endogenous expression and overexpression by transduction.

The term “expressible nucleic acid” means a nucleic acid coding for a proteinaceous molecule, an RNA molecule, or a DNA molecule.

The term “LIGASE” means a protein possessing E3 ubiquitin ligase activity. A preferred LIGASE comprises the type 1 transmembrane proteins. A most preferred LIGASE comprises the protein encoded by the polynucleotide sequence named, RNF128 (or alternatively as FLJ23516), including the naturally occurring transcript variants thereof.

The term “hybridization” means any process by which a strand of nucleic acid binds with a complementary strand through base pairing. The term “hybridization complex” refers to a complex formed between two nucleic acid sequences by virtue of the formation of hydrogen bonds between complementary bases. A hybridization complex may be formed in solution (e.g., C₀ₜ, or R₀ₜ analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed). The term “stringent conditions” refers to conditions that permit hybridization between polynucleotides and the claimed polynucleotides. Stringent conditions can be defined by salt concentration, the concentration of organic solvent, e.g., formamide, temperature, and other conditions well known in the art. In particular, reducing the concentration of salt, increasing the concentration of formamide, or raising the hybridization temperature can increase stringency.

The term “inhibit” or “inhibiting”, in relationship to the term “response” means that a response is decreased or prevented in the presence of a compound as opposed to in the absence of the compound.

The term “ligand” means an endogenous, naturally occurring molecule specific for an endogenous, naturally occurring receptor.

The term “modulates” means to change, or adjust, a process or function, or level of a material resulting from said process or function.

The term “pharmaceutically acceptable prodrugs” as used herein means the prodrugs of the compounds useful in the present invention, which are, within the scope of sound medical judgment, suitable for use in contact with the tissues of patients with undue toxicity, irritation, allergic response commensurate with a reasonable benefit/risk ratio, and effective for their intended use of the compounds of the invention. The term “prodrug” means a compound that is transformed in vivo to yield an effective compound useful in the present invention or a pharmaceutically acceptable salt, hydrate or solvate thereof. The transformation may occur by various mechanisms, such as through hydrolysis in blood. The compounds bearing metabolically cleavable groups have the advantage that they may exhibit improved bioavailability as a result of enhanced solubility and/or rate of absorption conferred upon the parent compound by virtue of the presence of the metabolically cleavable group, thus, such compounds act as pro-drugs. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8, A-C8, (1992); J. Pharm. Sci., 77, 285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference. An example of the prodrugs is an ester prodrug. “Ester prodrug” means a compound that is convertible in vivo by metabolic means (e.g., by hydrolysis) to an inhibitor compound according to the present invention. For example an ester prodrug of a compound containing a carboxy group may be convertible by hydrolysis in vivo to the corresponding carboxy group.

The term “pharmaceutically acceptable salts” refers to the non-toxic, inorganic and organic acid addition salts, and base addition salts, of compounds of the present invention. These salts can be prepared in situ during the final isolation and purification of compounds useful in the present invention.

The term “polynucleotide” means a polynucleic acid, in single or double stranded form, and in the sense or antisense orientation, complementary polynucleic acids that hybridize to a particular polynucleic acid under stringent conditions, and polynucleotides that are homologous in at least about 60 percent of its base pairs, and more preferably 70 percent of its base pairs are in common, most preferably 90 percent, and in a special embodiment 100 percent of its base pairs. The polynucleotides include polyribonucleic acids, polydeoxyribonucleic acids, and synthetic analogues thereof. The polynucleotides are described by sequences that vary in length, that range from about 10 to about 5000 bases, preferably about 100 to about 4000 bases, more preferably about 250 to about 2500 bases. A preferred polynucleotide embodiment comprises from about 10 to about 30 bases in length. A special embodiment of polynucleotide is the polyribonucleotide of from about 10 to about 22 nucleotides, more commonly described as small interfering RNAs (siRNAs). Another special embodiment are nucleic acids with modified backbones such as peptide nucleic acid (PNA), polysiloxane, and 2′-O-(2-methoxy)ethylphosphorothioate, or including non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection.

The term “polypeptide” relates to proteins, proteinaceous molecules, fractions of proteins (such as ligases), peptides and oligopeptides.

The term “solvate” means a physical association of a compound useful in this invention with one or more solvent molecules. This physical association includes hydrogen bonding. In certain instances the solvate is capable of isolation, for example when one or more solvent molecules are incorporated in the crystal lattice of the crystalline solid. “Solvate” encompasses both solution-phase and isolable solvates. Representative solvates include hydrates, ethanolates and methanolates.

The term “subject” includes humans and other mammals.

The term “effective amount” or “therapeutically effective amount” means that amount of a compound or agent that will elicit the biological or medical response of a subject that is being sought by a medical doctor or other clinician. In particular, with regard to treating an neuronal disorder, the term “effective amount ” is intended to mean that effective amyloid-beta precursor processing inhibiting amount of an compound or agent that will bring about a biologically meaningful decrease in the levels of amyloid beta peptide in the subject's brain tissue.

The term “treating” means an intervention performed with the intention of preventing the development or altering the pathology of, and thereby alleviating a disorder, disease or condition, including one or more symptoms of such disorder or condition. Accordingly, “treating” refers to both therapeutic treatment and prophylactic or preventative measures. Those in need of treating include those already with the disorder as well as those in which the disorder is to be prevented. The related term “treatment,” as used herein, refers to the act of treating a disorder, symptom, disease or condition, as the term “treating” is defined above.

The background of the present inventors' discovery is described briefly below.

Background of the LIGASE, RNF128:

The protein encoded by this the RNF128 polynucleotide is a type I transmembrane protein that localizes to the endocytic pathway. This protein contains a RING zinc-finger motif and has been shown to possess E3 ubiquitin ligase activity. Expression of this gene in retrovirally transduced T cell hybridoma significantly inhibits activation-induced IL2 and IL4 cytokine production. Induced expression of this gene was observed in anergic CD4(+) T cells, which suggested a role in the induction of anergic phenotype. Alternatively spliced transcript variants encoding distinct isoforms have been reported.

### REFERENCES

Soares, L., Seroogy, C., Skrenta, H., Anandasabapathy, N., Lovelace, P., Chung, C. D., Engleman, E. and Fathrnan, C. G., “Two isoforms of otubain 1 regulate T cell anergy via GRAIL”, JOURNAL Nat. Immunol. 5 (1), 45-54 (2004).
Anandasabapathy, N., Ford, G. S., Bloom, D., Holness, C., Paragas, V., Seroogy, C., Skrenta, H., Hollenhorst, M., Fathman, C. G. and Soares, L., “GRAIL: an E3 ubiquitin ligase that inhibits cytokine gene transcription is expressed in anergic CD4+ T cells”, JOURNAL Immunity 18 (4), 535-547 (2003).
   Applicants' Invention Based on LIGASE Relationship to Amyloid Beta Peptides

As noted above, the present invention is based on the present inventors' discovery that the LIGASE(s) are factors in the down-regulation and/or inhibition of amyloid beta precursor processing in mammalian cells, and that the induction or stimulation of the function of such polypeptides is effective in decreasing levels of the amyloid beta peptides.

The present inventors are unaware of any prior knowledge linking LIGASEs, and more particularly RNF128 (Table 1 below), and amyloid beta peptide formation and secretion. As discussed in more detail in the Experimental section below, the present inventors demonstrate that the increased expression of RNF128 reduces, amyloid beta 1-42, x-42, 1-40 and 1-y in the conditioned medium of transduced cells. The present invention is based on these findings and the recognition that these LIGASEs are putative drug targets for Alzheimer's disease, particularly tin view of the expression of RNF128 in the tissue of the human central nervous system.

TABLE 1

| Accession | | | SEQ ID NOs: | |
|---|---|---|---|---|
| No. | Description | Code | DNA | Protein |
| NM_194463 | ring finger protein 128 | RNF128 | 1 | 2 |

One aspect of the present invention is a method based on the aforesaid discovery for identifying a compound that inhibits the aberrant processing of amyloid-beta precursor protein in a mammalian cell, and may therefore be useful in reducing undesirable amyloid beta peptide levels in a subject. The present method comprises contacting a drug candidate compound with a LIGASE polypeptide, or a fragment of said polypeptide, and measuring a compound-polypeptide property related to the production of amyloid-beta protein. The “compound-polypeptide property” is a measurable phenomenon chosen by the person of ordinary skill in the art, and based on the recognition that LIGASE activation and deactivation is a causative factor in the deactivation and activation, respectively, of amyloid beta protein precursor processing, and an decrease and increase, respectively, of amyloid beta peptide levels. The measurable property may range from the binding affinity for a peptide domain of the LIGASE polypeptide, to the level of amyloid beta peptide secreted by the mammalian cell contacted with the compound.

Depending on the choice of the skilled artisan, the present assay method may be designed to function as a series of measurements, each of which is designed to determine whether the drug candidate compound is indeed acting on the LIGASE to amyloid beta peptide pathway. For example, an assay designed to determine the binding affinity of a compound to the LIGASE, or fragment thereof, may be necessary, but not sufficient, to ascertain whether the test compound would be useful for reducing amyloid beta peptide levels when administered to a subject. Nonetheless, such binding information would be useful in identifying a set of test compounds for use in an assay that would measure a different property, further down the biochemical pathway. Such second assay may be designed to confirm that the test compound, having binding affinity for a LIGASE peptide, actually up-regulates or induces, as an agonist, LIGASE function in a mammalian cell. Confirming that the assay system itself is not being affected directly and not the LIGASE pathway may further validate the assay. In this latter regard, suitable controls should always be in place to insure against false positive readings.

The order of taking these measurements is not believed to be critical to the practice of the present invention, which may be practiced in any order. For example, one may first perform a screening assay of a set of compounds for which no information is known respecting the compounds' binding affinity for LIGASE. Alternatively, one may screen a set of compounds identified as having binding affinity for a LIGASE peptide domain, or a class of compounds identified as an agonist of a LIGASE. It is not essential to know the binding affinity for LIGASE due to the possible compound interaction in the intra-membrane domain of the LIGASE polypeptide, which domain conformation may not be possible to reproduce in an affinity experiment. However, for the present assay to be meaningful to the ultimate use of the drug candidate compounds, a measurement of the ultimate amyloid beta peptide levels is necessary. Validation studies including controls, and measurements of binding affinity to LIGASE are nonetheless useful in identifying a compound useful in any therapeutic or diagnostic application.

The present assay method may be practiced in vitro, using one or more of the LIGASE proteins, or fragments thereof, or membrane preparations made from cells transduced with vectors over-expressing the LIGASE polypeptides. The amino acid sequences of the most preferred LIGASEs, and useful fragments thereof are found in SEQ ID NO: 2 and 3-5. The binding affinity of the compound with the polypeptide can be measured by methods known in the art, such as using surface plasmon resonance biosensors (Biacore), by saturation binding analysis with a labeled compound (e.g. Scatchard and Lindmo analysis), by differential UV spectrophotometer, fluorescence polarization assay, Fluorometric Imaging Plate Reader (FLIPR®) system, Fluorescence resonance energy transfer, and Bioluminescence resonance energy transfer. The binding affinity of compounds can also be expressed in dissociation constant (Kd) or as IC50 or EC50. The IC50 represents the concentration of a compound that is required for 50% inhibition of binding of another ligand to the polypeptide. The EC50 represents the concentration required for obtaining 50% of the maximum effect in any assay that measures receptor function. The dissociation constant, Kd, is a measure of how well a ligand binds to the polypeptide, it is equivalent to the ligand concentration required to saturate exactly half of the binding-sites on the polypeptide. Compounds with a high affinity binding have low Kd, IC50 and EC50 values, i.e. in the range of 100 nM to 1 pM; a moderate to low affinity binding relates to a high Kd, IC50 and EC50 values, i.e. in the micromolar range.

The present assay method may also be practiced in a cellular assay, A host cell expressing a LIGASE polypeptide can be a cell with endogenous expression of the polypeptide or a cell over-expressing the polypeptide e.g. by transduction. The addition of an agonist further stimulates LIGASE thereby further decreasing secreted amyloid beta peptides. The LIGASE polypeptides, when over expressed or activated, reduce the level of secreted amyloid beta peptides.

The present invention further relates to a method for identifying a compound that inhibits aberrant amyloid-beta precursor protein processing in a mammalian cell comprising:
(a) contacting a compound with a polypeptide comprising an amino acid sequence selected from the group consisting of SEQ ID NO: 2 and 3-5,
(b) determining the binding affinity of the compound to the polypeptide,
(c) contacting a population of mammalian cells expressing said polypeptide with the compound that exhibits a binding affinity of at least 10 micromolar, and
(d) identifying the compound that inhibits the aberrant amyloid-beta precursor protein processing in the cells.

A further embodiment of the present invention relates a method to identify a compound that inhibits the aberrant amyloid-beta precursor protein processing in a cell, wherein the activity level of the LIGASE polypeptide is measured by determining the level of amyloid beta peptides. The levels of these peptides may be measured with specific ELISAs using antibodies specifically recognizing the different amyloid beta peptide species (see e.g. EXAMPLE 1). Secretion of the various amyloid beta peptides may also be measured using antibodies that bind all peptides. Levels of amyloid beta peptides can also be measured by mass spectrometry analysis.

For high-throughput purposes, libraries of compounds may be used such as antibody fragment libraries, peptide phage display libraries, peptide libraries (e.g. LOPAP™, Sigma Aldrich), lipid libraries (BioMol), synthetic compound libraries (e.g. LOPACT™, Sigma Aldrich) or natural compound libraries (Specs, TimTec).

Preferred drug candidate compounds are low molecular weight compounds. Low molecular weight compounds, i.e. with a molecular weight of 500 Dalton or less, are likely to have good absorption and permeation in biological systems and are consequently more likely to be successful drug candidates than compounds with a molecular weight above 500 Dalton (Lipinski et al. (1997)). Peptides comprise another preferred class of drug candidate compounds. Peptides may be excellent drug candidates and there are multiple examples of commercially valuable peptides such as fertility hormones and platelet aggregation inhibitors. Natural compounds are another preferred class of drug candidate compound. Such compounds are found in and extracted from natural sources, and which may thereafter be synthesized.

Another preferred class of drug candidate compounds is an antibody binding ligand. The present invention also provides antibodies directed against the extracellular domains of the LIGASE. These antibodies should specifically bind to one or more of the extra-cellular domains of the LIGASE, or as described further below, engineered to be endogenously produced to bind to the intra-cellular LIGASE domain. These antibodies may be monoclonal antibodies or polyclonal antibodies. The present invention includes chimeric, single chain, and humanized antibodies, as well as FAb fragments and the products of a FAb expression library, and Fv fragments and the products of an Fv expression library. Most preferred antibodies include only the binding domain of the antibody, which domain would be prepared only to bind the LIGASE to set the LIGASE to an “active” constitutive state, and not to target the cell for immunological attack.

In certain embodiments, polyclonal antibodies may be used in the practice of the invention. The skilled artisan knows methods of preparing polyclonal antibodies. Polyclonal antibodies can be raised in a mammal, for example, by one or more injections of an immunizing agent and, if desired, an adjuvant. Typically, the immunizing agent and/or adjuvant is injected in the mammal by multiple subcutaneous or intraperitoneal injections. Antibodies may also be generated against the intact LIGASE protein or polypeptide, or against a fragment such as its extracellular domain peptides, derivatives including conjugates, or other epitope of the LIGASE protein or polypeptide, such as the LIGASE embedded in a cellular membrane, or a library of antibody variable regions, such as a phage display library.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). One skilled in the art without undue experimentation may select the immunization protocol.

In some embodiments, the antibodies may be monoclonal antibodies. Monoclonal antibodies may be prepared using methods known in the art. The monoclonal antibodies of the present invention may be “humanized” to prevent the host from mounting an immune response to the antibodies. A “humanized antibody” is one in which the complementarity determining regions (CDRs) and/or other portions of the light and/or heavy variable domain framework are derived from a non-human immunoglobulin, but the remaining portions of the molecule are derived from one or more human immunoglobulins. Humanized antibodies also include antibodies characterized by a humanized heavy chain associated with a donor or acceptor unmodified light chain or a chimeric light chain, or vice versa. The humanization of antibodies may be accomplished by methods known in the art (see, e.g. Mark and Padlan, (1994) “Chapter 4. Humanization of Monoclonal Antibodies”, The Handbook of Experimental Pharmacology Vol. 113, Springer-Verlag, New York). Transgenic animals may be used to express humanized antibodies.

Human antibodies can also be produced using various techniques known in the art, including phage display libraries (Hoogenboom and Winter, (1991) J. Mol. Biol. 227:381-8; Marks et al. (1991). J. Mol. Biol. 222:581-97). The techniques of Cole, et al. and Boerner, et al. are also available for the preparation of human monoclonal antibodies (Cole, et al. (1985) Monoclonal Antibodies and Cancer Therapy, Alan R. Liss, p. 77; Boerner, et al (1991). J. Immunol., 147(1):86-95).

Techniques known in the art for the production of single chain antibodies can be adapted to produce single chain antibodies to the LIGASE polypeptides. The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively; the relevant cysteine residues are substituted with another amino acid residue or are deleted so as to prevent cross-linking.

Bispecific antibodies are monoclonal, preferably human or humanized, antibodies that have binding specificities for at least two different antigens and preferably for a cell-surface protein or receptor or receptor subunit. In the present case, one of the binding specificities is for one extracellular domain of the LIGASE, the other one is for another extracellular domain of the LIGASE.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, (1983) Nature 305:537-9). Because of the random assortment of immunoglobulin heavy and light chains, these hybridomas (quadromas) produce a potential mixture of ten different antibody molecules, of which only one has the correct bispecific structure. Affinity chromatography steps usually accomplish the purification of the correct molecule. Similar procedures are disclosed in Trauneeker, et al. (1991) EMBO J. 10:3655-9.

According to another preferred embodiment, the assay method comprises using a drug candidate compound identified as having a binding affinity for LIGASE, and/or has already been identified as having agonist activity vis-à-vis one or more LIGASE.

Another aspect of the present invention relates to a method for reducing aberrant amyloid-beta precursor protein processing in a mammalian cell, comprising by contacting said cell with an DNA expression agent that encodes a LIGASE polypeptide. A particular embodiment preferably comprises polynucleotide sequence complementary to, or engineered from, a naturally occurring polynucleotide sequence encoding a polypeptide comprising an amino acid sequence selected from the group consisting of SEQ ID NO: 2.

The present invention also relates to compositions, and methods using said compositions, comprising a DNA expression vector capable of expressing a polynucleotide as described herein.

The DNA expression vector comprises the polynucleotide expressing the agent is operably linked to signals enabling expression of the nucleic acid sequence and is introduced into a cell utilizing, preferably, recombinant vector constructs, which will express the nucleic acid once the vector is introduced into the cell. A variety of viral-based systems are available, including adenoviral, retroviral, adeno-associated viral, lentiviral, herpes simplex viral or a sendaviral vector systems, and all may be used to introduce and express polynucleotide sequence for the DNA expression agents in target cells.

Preferably, the viral vectors used in the methods of the present invention are replication defective. Such replication defective vectors will usually lack at least one region that is necessary for the replication of the virus in the infected cell. These regions can either be eliminated (in whole or in part), or be rendered non-functional by any technique known to a person skilled in the art. These techniques include the total removal, substitution, partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome, which are necessary for encapsidating, the viral particles.

In a preferred embodiment, the viral element is derived from an adenovirus. Preferably, the vehicle includes an adenoviral vector packaged into an adenoviral capsid, or a functional part, derivative, and/or analogue thereof. Adenovirus biology is also comparatively well known on the molecular level. Many tools for adenoviral vectors have been and continue to be developed, thus making an adenoviral capsid a preferred vehicle for incorporating in a library of the invention. An adenovirus is capable of infecting a wide variety of cells. However, different adenoviral serotypes have different preferences for cells. To combine and widen the target cell population that an adenoviral capsid of the invention can enter in a preferred embodiment, the vehicle includes adenoviral fiber proteins from at least two adenoviruses. Preferred adenoviral fiber protein sequences are serotype 17, 45 and 51. Techniques or construction and expression of these chimeric vectors are disclosed in US Published Patent Applications 20030180258 and 20040071660, hereby incorporated by reference.

In a preferred embodiment, the nucleic acid derived from an adenovirus includes the nucleic acid encoding an adenoviral late protein or a functional part, derivative, and/or analogue thereof. An adenoviral late protein, for instance an adenoviral fiber protein, may be favorably used to target the vehicle to a certain cell or to induce enhanced delivery of the vehicle to the cell. Preferably, the nucleic acid derived from an adenovirus encodes for essentially all adenoviral late proteins, enabling the formation of entire adenoviral capsids or functional parts, analogues, and/or derivatives thereof. Preferably, the nucleic acid derived from an adenovirus includes the nucleic acid encoding adenovirus E2A or a functional part, derivative, and/or analogue thereof. Preferably, the nucleic acid derived from an adenovirus includes the nucleic acid encoding at least one E4-region protein or a functional part, derivative, and/or analogue thereof, which facilitates, at least in part, replication of an adenoviral derived nucleic acid in a cell. The adenoviral vectors used in the examples of this application are exemplary of the vectors useful in the present method of treatment invention.

Certain embodiments of the present invention use retroviral vector systems. Retroviruses are integrating viruses that infect dividing cells, and their construction is known in the art. Retroviral vectors can be constructed from different types of retrovirus, such as, MoMuLV (“murine Moloney leukemia virus” MSV (“murine Moloney sarcoma virus”), HaSV (“Harvey sarcoma virus”); SNV (“spleen necrosis virus”); RSV (“Rous sarcoma virus”) and Friend virus. Lentiviral vector systems may also be used in the practice of the present invention. Retroviral systems and herpes virus system may be preferred vehicles for transfection of neuronal cells.

In other embodiments of the present invention, adeno-associated viruses (“AAV”) are utilized. The AAV viruses are DNA viruses of relatively small size that integrate, in a stable and site-specific manner, into the genome of the infected cells. They are able to infect a wide spectrum of cells without inducing any effects on cellular growth, morphology or differentiation, and they do not appear to be involved in human pathologies.

In the vector construction, the polynucleotide agents of the present invention may be linked to one or more regulatory regions. Selection of the appropriate regulatory region or regions is a routine matter, within the level of ordinary skill in the art. Regulatory regions include promoters, and may include enhancers, suppressors, etc.

Promoters that may be used in the expression vectors of the present invention include both constitutive promoters and regulated (inducible) promoters. The promoters may be prokaryotic or eukaryotic depending on the host. Among the prokaryotic (including bacteriophage) promoters useful for practice of this invention are lac, lacZ, T3, T7, lambda Pr P₁, and trp promoters. Among the eukaryotic (including viral) promoters useful for practice of this invention are ubiquitous promoters (e.g. HPRT, vimentin, actin, tubulin), intermediate filament promoters (e.g. desmin, neurofilaments, keratin, GFAP), therapeutic gene promoters (e.g. MDR type, CFTR, factor VIII), tissue-specific promoters (e.g. actin promoter in smooth muscle cells, or Flt and Flk promoters active in endothelial cells), including animal transcriptional control regions, which exhibit tissue specificity and have been utilized in transgenic animals: elastase I gene control region which is active in pancreatic acinar cells (Swift, et al. (1984) Cell 38:639-46; Ornitz, et al. (1986) Cold Spring Harbor Symp. Quant. Biol. 50:399-409; MacDonald, (1987) Hepatology 7:425-515); insulin gene control region which is active in pancreatic beta cells (Hanahan, (1985) Nature 315:115-22), immunoglobulin gene control region which is active in lymphoid cells (Grosschedl, et al. (1984) Cell 38:647-58; Adames, et al. (1985) Nature 318:533-8; Alexander, et al. (1987) Mol. Cell. Biol. 7:1436-44), mouse mammary tumor virus control region which is active in testicular, breast, lymphoid and mast cells (Leder, et al. (1986) Cell 45:485-95), albumin gene control region which is active in liver (Pinkert, et al. (1987) Genes and Devel. 1:268-76), alpha-fetoprotein gene control region which is active in liver (Krumlauf, et al. (1985) Mol. Cell. Biol., 5:1639-48; Hammer, et al. (1987) Science 235:53-8), alpha 1-antitrypsin gene control region which is active in the liver (Kelsey, et al. (1987) Genes and Devel., 1: 161-71), beta-globin gene control region which is active in myeloid cells (Mogram, et al. (1985) Nature 315:338-40; Kollias, et al. (1986) Cell 46:89-94), myelin basic protein gene control region which is active in oligodendrocyte cells in the brain (Readhead, et al. (1987) Cell 48:703-12), myosin light chain-2 gene control region which is active in skeletal muscle (Sani, (1985) Nature 314.283-6), and gonadotropic releasing hormone gene control region which is active in the hypothalamus (Mason, et al. (1986) Science 234:1372-8).

Other promoters which may be used in the practice of the invention include promoters which are preferentially activated in dividing cells, promoters which respond to a stimulus (e.g. steroid hormone receptor, retinoic acid receptor), tetracycline-regulated transcriptional modulators, cytomegalovirus immediate-early, retroviral LTR, metallothionein, SV-40, E1 a, and MLP promoters.

The vectors may also include other elements, such as enhancers, repressor systems,, and localization signals. A membrane localization signal is a preferred element when expressing a sequence encoding an intracellular binding protein, which functions by contacting the intracellular domain of the LIGASE and is most effective when the vector product is directed to the inner surface of the cellular membrane, where its target resides. Membrane localization signals are well known to persons skilled in the art. For example, a membrane localization domain suitable for localizing a polypeptide to the plasma membrane is the C-terminal sequence CaaX for farnesylation (where “a” is an aliphatic amino acid residue, and “X” is any amino acid residue, generally leucine), for example, Cysteine-Alanine-Alanine-Leucine, or Cysteine-Isoleucine-Valine-Methionine. Other membrane localization signals include the putative membrane localization sequence from the C-terminus of Bcl-2 or the C-terminus of other members of the Bcl-2 family of proteins.

Additional vector systems include the non-viral systems that facilitate introduction of polynucleotide agents into a patient. For example, a DNA vector encoding a desired sequence can be introduced in vivo by lipofection. Synthetic cationic lipids designed to limit the difficulties encountered with liposome-mediated transfection can be used to prepare liposomes for in vivo transfection of a gene encoding a marker (Feigner, et. al. (1987) Proc. Natl. Acad. Sci. USA 84:7413-7); see Mackey, et al. (1988) Proc. Natl. Acad. Sci. USA 85:8027-31; Ulmer, et al. (1993) Science 259:1745-8). The use of cationic lipids may promote encapsulation of negatively charged nucleic acids, and also promote fusion with negatively charged cell membranes (Felgner and Ringold, (1989) Nature 337:387-8).

Particularly useful lipid compounds and compositions for transfer of nucleic acids are described in International Patent Publications WO 95/18863 and WO 96/17823, and in U.S. Pat. No. 5,459,127. The use of lipofection to introduce exogenous genes into the specific organs in vivo has certain practical advantages and directing transfection to particular cell types would be particularly advantageous in a tissue with cellular heterogeneity, for example, pancreas, liver, kidney, and the brain. Lipids may be chemically coupled to other molecules for the purpose of targeting. Targeted peptides, e.g., hormones or neurotransmitters, and proteins for example, antibodies, or non-peptide molecules could be coupled to liposomes chemically. Other molecules are also useful for facilitating transfection of a nucleic acid in vivo, for example, a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce a DNA vector in vivo as a naked DNA plasmid (see U.S. Pat. Nos. 5,693,622, 5,589,466 and 5,580,859). Naked DNA vectors for therapeutic purposes can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wilson, et al. (1992) J. Biol. Chem. 267:963-7; Wu and Wu, (1988) J. Biol. Chem. 263:14621-4; Hartmut, et al. Canadian Patent Application No. 2,012,311, filed Mar. 15, 1990; Williams, et al (1991). Proc. Natl. Acad. Sci. USA 88:2726-30). Receptor-mediated DNA delivery approaches can also be used (Curiel, et al. (1992) Hum. Gene Ther. 3:147-54; Wu and Wu, (1987) J. Biol. Chem. 262:4429-32).

The present invention also provides biologically compatible compositions comprising the compounds identified as agonists of LIGASE, and the DNA expression-agents as described hereinabove.

A biologically compatible composition is a composition, that may be solid, liquid, gel, or other form, in which the compound, polynucleotide, vector, and antibody of the invention is maintained in an active form, e.g., in a form able to effect a biological activity. For example, a compound of the invention would have agonist activity on the LIGASE; a nucleic acid would be able to replicate, translate a message, or hybridize to a complementary mRNA of a LIGASE; a vector would be able to transfect a target cell and express the mRNA of a LIGASE as described hereinabove; an antibody would bind a LIGASE polypeptide domain.

A preferred biologically compatible composition is an aqueous solution that is buffered using, e.g., Tris, phosphate, or HEPES buffer, containing salt ions. Usually the concentration of salt ions are similar to physiological levels. Biologically compatible solutions may include stabilizing agents and preservatives. In a more preferred embodiment, the biocompatible composition is a pharmaceutically acceptable composition. Such compositions can be formulated for administration by topical, oral, parenteral, intranasal, subcutaneous, and intraocular, routes. Parenteral administration is meant to include intravenous injection, intramuscular injection, intraarterial injection or infusion techniques. The composition may be administered parenterally in dosage unit formulations containing standard, well known non-toxic physiologically acceptable carriers, adjuvants and vehicles as desired.

A particularly preferred embodiment of the present composition invention is a cognitive-enhancing pharmaceutical composition comprising a therapeutically effective amount of an DNA expression agent as described hereinabove, in admixture with a pharmaceutically acceptable carrier. Another preferred embodiment is a pharmaceutical composition for the treatment or prevention of a condition involving cognitive impairment or a susceptibility to the condition, comprising an effective amyloid beta peptide inhibiting amount of a LIGASE agonist its pharmaceutically acceptable salts, hydrates, solvates, or prodrugs thereof in admixture with a pharmaceutically acceptable carrier.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical compositions for oral use can be prepared by combining active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl-cellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinyl-pyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Preferred sterile injectable preparations can be a solution or suspension in a non-toxic parenterally acceptable solvent or diluent. Examples of pharmaceutically acceptable carriers are saline, buffered saline, isotonic saline (e.g. monosodium or disodium phosphate, sodium, potassium; calcium or magnesium chloride, or mixtures of such salts), Ringer's solution, dextrose, water, sterile water, glycerol, ethanol, and combinations thereof 1,3-butanediol and sterile fixed oils are conveniently employed as solvents or suspending media. Any bland fixed oil can be employed including synthetic mono- or di-glycerides. Fatty acids such as oleic acid also find use in the preparation of injectables.

The composition medium can also be a hydrogel, which is prepared from any biocompatible or non-cytotoxic homo- or hetero-polymer, such as a hydrophilic polyacrylic acid polymer that can act as a drug absorbing sponge. Certain of them, such as, in particular, those obtained from ethylene and/or propylene oxide are commercially available. A hydrogel can be deposited directly onto the surface of the tissue to be treated, for example during surgical intervention.

Embodiments of pharmaceutical compositions of the present invention comprise a replication defective recombinant viral vector encoding the polynucleotide expression agent of the present invention and a transfection enhancer, such as poloxamer. An example of a poloxamer is Poloxamer 407, which is commercially available (BASF, Parsippany, N.J.) and is a non-toxic, biocompatible polyol. A poloxamer impregnated with recombinant viruses may be deposited directly on the surface of the tissue to be treated, for example during a surgical intervention. Poloxamer possesses essentially the same advantages as hydrogel while having a lower viscosity.

The active DNA expression agents may also be entrapped in microcapsules prepared, for example, by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1980) 16th edition, Osol, A. Ed.

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™. (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The present invention also provides methods of inhibiting the aberrant processing of amyloid-beta precursor protein in a subject suffering or susceptible to the abnormal processing of said protein, which comprise the administration to said subject of a therapeutically effective amount of an DNA expression agent of the invention. Another aspect of the present method invention is the treatment or prevention of a condition involving cognitive impairment or a susceptibility to the condition. A special embodiment of this invention is a method wherein the condition is Alzheimer's disease.

As defined above, therapeutically effective dose means that amount of protein, polynucleotide, peptide, or agonist, which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The pharmaceutical compositions according to this invention may be administered to a subject by a variety of methods. They may be added directly to target tissues, complexed with cationic lipids, packaged within liposomes, or delivered to target cells by other methods known in the art. Localized administration to the desired tissues may be done by catheter, infusion pump or stent. The DNA, DNA/vehicle complexes, or the recombinant virus particles are locally administered to the site of treatment. Alternative routes of delivery include, but are not limited to, intravenous injection, intramuscular injection, subcutaneous injection, aerosol inhalation, oral (tablet or pill form), topical, systemic, ocular, intraperitoneal and/or intrathecal delivery. Examples of ribozyme delivery and administration are provided in Sullivan et al. WO 94/02595.

Antibodies according to the invention may be delivered as a bolus only, infused over time or both administered as a bolus and infused over time. Those skilled in the art may employ different formulations for polynucleotides than for proteins. Similarly, delivery of polynucleotides or polypeptides is specific to particular cells, conditions, locations, etc.

As discussed hereinabove, recombinant viruses may be used to introduce DNA encoding polynucleotide agents useful in the present invention. Recombinant viruses according to the invention are generally formulated and administered in the form of doses of between about 10⁴ and about 10¹⁴ pfu. In the case of AAVs and adenoviruses, doses of from about 10⁶ to about 10¹¹ pfu are preferably used. The term pfu (“plaque-forming unit”) corresponds to the infective power of a suspension of virions and is determined by infecting an appropriate cell culture and measuring the number of plaques formed. The techniques for determining the pfu titre of a viral solution are well documented in the prior art.

Still another aspect or the invention relates to a method for diagnosing a pathological condition involving cognitive impairment or a susceptibility to the condition in a subject, comprising determining the amount of polypeptide comprising an amino acid sequence of SEQ ID NO: 2, or fragment thereof, in a biological sample, and comparing the amount with the amount of the polypeptide in a healthy subject, wherein an increase of the amount of polypeptide compared to the healthy subject is indicative of the presence of the pathological condition.

### Experimental Section

### EXAMPLE 1

### Assay for Novel Drug Targets to Inhibit Amyloid Beta 1-42 Levels

To identify novel drug targets that change the APP processing, stable cell lines over expressing APP are made by transfecting Hek293 or SH-SY5Y cells with APP770 wt cDNA cloned into pcDNA3.1, followed by selection with G418 for 3 weeks. At this time point colonies are picked and stable clones are expanded and tested for their secreted amyloid-beta peptide levels. The cell lines designated as “Hek293 APPwt” and “SH-SY5Y APPwt” are used in the assays.

Hek293 APPwt Assay: Cells seeded in collagen-coated plates at a cell density of 15000 cells/well (384 well plate) in DMEM (10% FBS), are infected 24 h later with 1 μl or 0.2 μl of adenovirus (corresponding to an average multiplicity of infection (MOI) of 120 and 24 respectively). The following day, the virus is washed away and DMEM (25 mM Hepes; 10% FBS) is added to the cells. Amyloid-beta peptides are allowed to accumulate during 24h.

SH-SY5Y APPwt Assay: Cells are seeded in collagen-coated plates at a cell density of 15000 cells/well (384 well plate) in Dulbecco's MEM with Glutamax I+15% FBS HI+non-essential amino acids+Geneticin 500 μg/ml. The cells are differentiated towards the neuronal phenotype by adding 9-cis retinoic acid to a final concentration of 1 μM on day 1, day 3, day 5 and day 8. On day 9, the cells are infected with 1 μl of adenovirus (corresponding to an average multiplicity of infection (MOI) of 120 respectively). The following day, the virus is washed away and DMEM 25 mM Hepes 10% FBS is added to the cells. Amyloid beta peptides are allowed to accumulate for 24 h.

ELISA: The ELISA plate is prepared by coating with a capture antibody (JRF/cAbeta42/26) (the antibody recognizes a specific epitope on the C-terminus of Abeta 1-42; obtained from M Mercken, Johnson and Johnson Pharmaceutical Research and Development, B-2340 Beerse, Belgium) overnight in buffer 42 (Table 2) at a concentration of 2.5 μg/ml. The excess capture antibody is washed away the next morning with PBS and the ELISA plate is then blocked overnight with casein buffer (see Table 2) at 4° C. Upon removal of the blocking buffer, 30 μl of the sample is transferred to the ELISA plate and incubated overnight at 4° C. After extensive washing with PBS-Tween20 and PBS, 30 μl of the horseradish peroxidase (HRP) labeled detection antibody (Peroxidase Labeling Kit, Roche), JRF/AbetaN/25-HRP (obtained from M Mercken, Johnson and Johnson Pharmaceutical Research and Development, B-2340 Beerse, Belgium) is diluted 1/5000 in buffer C (see Table 2) and added to the wells for another 2 h. Following the removal of excess detection antibody by a wash with PBS-Tween20 and PBS, HRP activity is detected via addition of luminol substrate (Roche), which is converted into a chemiluminescent signal by the HRP enzyme.

In addition, for the SH-SY5Y APPwt assay, the samples are also analyzed in an amyloid beta x-42 ELISA. This ELISA detects all amyloid beta peptide species ending at position 42, comprising 1-42, 11-42 and 17-42 (p3), which originate respectively from BACE activity at position 1 and 11, and alpha secretase activity at position 17. Thus, in addition to the amyloidogenic pathway, the non-amyloidogenic pathway is also monitored. The protocol for the Abeta x-42 ELISA is identical to the protocol for the Abeta 1-42 ELISA, except that a HRP labeled 4G8 antibody (Signet; the antibody recognizes a specific epitope in the center of the Abeta peptides) is used as detection antibody.

TABLE 2
Buffers And Solutions Used For ELISA

| | |
|---|---|
| Buffer 42 | 30 mM NaHCO₃, 70 mM Na₂CO₃, 0.05% NaN₃, pH9.6 |
| Casein | 0.1% casein in PBS 1× |
| buffer | |
| EC Buffer | 20 mM sodium phosphate, 2 mM EDTA, 400 mM NaCl, |
| | 0.2% BSA, 0.05% CHAPS, 0.4% casein, 0.05% NaN₃, pH7 |
| Buffer C | 20 mM sodium phosphate, 2 mM EDTA, 400 mM NaCl, 1% |
| | BSA, pH7 |
| PBS 10× | 80 g NaCl + 2 g KCl + 11.5 g Na₂HPO₄.7H₂O + 2 g KH₂PO₄ |
| | in 1 1 milli Q, pH 7.4 |
| PBST | PBS 1× with 0.05% Tween 20 |

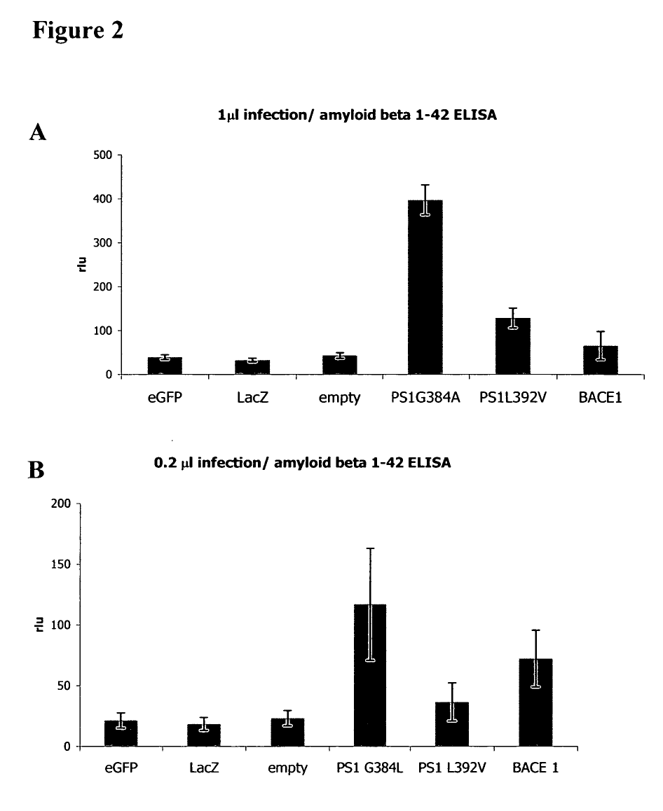

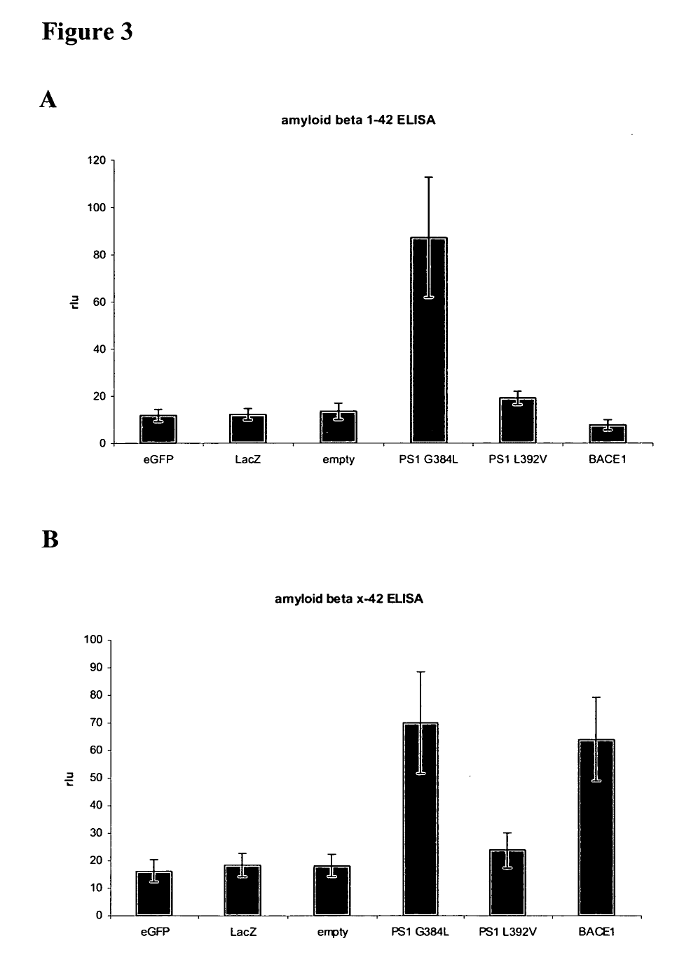

To validate the assay, the effect of adenoviral over expression with random titer of two clinical PS1 mutants and BACE on amyloid beta 1-42 production is evaluated in the Hek293 APPwt cells. As is shown in FIG. 2, all PS1 and BACE constructs induce amyloid beta 1-42 levels as expected. As is shown in FIG. 3, adenoviral overexpression of the clinical PS1 mutants in the SH-SY5Y APPwt cells also yield a significant induction of amyloid beta 1-42 levels. However, since overexpression of BACE in the SH-SY5Y APPwt cells do not result in an induction of amyloid beta 1-42 levels, amyloid beta x-42 levels are determined and show a clear induction.

An adenoviral cDNA library was constructed as follows. DNA fragments covering the full coding region of the target candidate genes are amplified by PCR from a pooled placental and fetal liver cDNA library (InvitroGen). All fragments are cloned into an adenoviral vector as described in U.S. Pat. No. 6,340,595, the contents of which are herein incorporated by reference, and subsequently adenoviruses are made harboring the corresponding cDNAs. The screen types using these libraries are presented in Table 3.

TABLE 3

| Screen number | Cell type | ELISA | Adenoviral library |
|---|---|---|---|
| H25 | Hek293 APPwt | Abeta 1-42 | KI-library |
| H22 | SH-SY5Y APPwt | Abeta 1-42 | KI-library |
| H28 | SH-SY5Y APPwt | Abeta x-42 | KI-library |

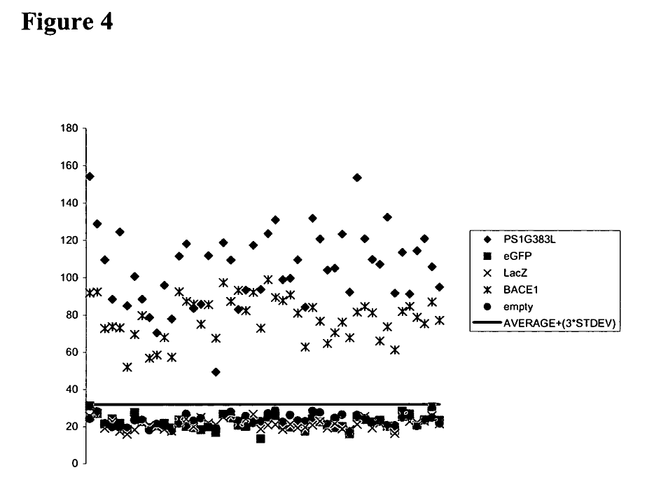

Activators of amyloid beta production are selected by calculating the average and standard deviation of all data points during the screening run (i.e. all plates processed in one week) and applying the formula AVERAGE+(N×STDEV) to calculate the cut off value (N is determined individually for every screen and is indicated in TABLES 4A, 4B, 4C, 4D, 4E, 4F, which present the results of the screenings). All cDNAs scoring higher then the cut off value are considered as positives and thus modulate amyloid beta 1-42 levels. This is validated by infecting Hek293APPwt cells with a control plate containing PS1 G384L, BACE1 and eGFP, empty and LacZ adenoviruses. The average and standard deviation are calculated based upon the negative controls. Applying the cut off (AVERAGE+(3×STDEV)) reveals that all positive controls are identified as positive data points (FIG. 4). Repressors of the amyloid beta production are selected in a similar way, except that the cDNAs have to score lower than the cut off value determined by the formula AVERAGE−(N×STDEV). The same procedure applies for the SH-SY5Y APPwt cells. One of the selected activators during the screen was APP, underscoring the relevance of the identified hits.

Four data points are obtained for every screen. A cDNA is considered a hit when at least 2 data points score positive out of 4. Blank boxes indicate that the screen was not performed for that specific cDNA. [Act is activator, Rep is repressor. A hit is indicated as the number 1. A negative data point is indicated as “−”. PS and RS represent respectively primary screen and rescreen].

TABLE 4 A

| screen | H25 | | | | | | | |
|---|---|---|---|---|---|---|---|---|
| infection | 0.25 μl | | | | 1 μl | | | |
| N for Act | 3 | | | | 3 | | | |
| N for Rep | −1.6 | | | | −1.6 | | | |
| cDNA | PS | | RS | | PS | | RS | |
| FLJ23516 (RNF128) | −1.803 | −1.697 | −2.421 | −2.545 | −2.114 | −2.15 | −2.348 | −2.449 |
| | 1 | 1 | 1 | 1 | 1 | 1 | 1 | 1 |
| MAP2K6 | 4.048 | 4.615 | 2.878 | 1.854 | 2.254 | 2.943 | 1.474 | 2.606 |
| | 1 | 1 | 0 | 0 | 0 | 0 | 0 | 0 |
| APP | 4.417 | 5.43 | 4.813 | 3.219 | 5.479 | 3.515 | 1.473 | 3.729 |
| | 1 | 1 | 1 | 1 | 1 | 1 | 0 | 1 |
| GZMM | 2.783 | 3.979 | 3.378 | 2.252 | 2.951 | 4.46 | 0.312 | 2.75 |
| | 0 | 1 | 1 | 0 | 0 | 1 | 0 | 0 |
| USP2 | 0.544 | −0.013 | 1.832 | 1.795 | 2.971 | 3.869 | 1.473 | 3.034 |
| | 0 | 0 | 0 | 0 | 0 | 1 | 0 | 1 |
| ENSG00000117094 | 2.875 | 2.898 | 4.554 | 4.65 | 3.286 | 3.433 | 4.146 | 4.091 |
| | 0 | 0 | 1 | 1 | 1 | 1 | 1 | 1 |
| ADAMTS4 | 0.128 | 0.522 | 0.696 | 0.243 | −0.419 | −0.543 | −0.486 | −0.672 |
| | 0 | 0 | 0 | 0 | 0 | 0 | 0 | 0 |
| USP21 | 5.118 | 6.018 | 4.468 | 1.449 | 1.481 | 6.015 | 6.658 | 3.401 |
| | 1 | 1 | 1 | 0 | 0 | 1 | 1 | 0 |
| LTB4R | −2.903 | −2.492 | | | −2.094 | −2.121 | −3.419 | −3.217 |
| | 0 | 0 | | | 1 | 1 | 1 | 1 |

TABLE 4B

| screen | H22 | H28 |
|---|---|---|
| infection | 1 μl | 1 μl |
| N for Act | 3 | 3 |
| N for Rep | −1.6 | −1.6 |

| cDNA | PS | | RS | | PS | | RS | |
|---|---|---|---|---|---|---|---|---|
| FLJ23516 | −2.091 | −2.397 | −2.339 | −2.611 | −1.51 | −1.376 | −2.571 | −3.09 |
| (RNF128) | 1 | 1 | 0 | 1 | 0 | 0 | 1 | 1 |
| MAP2K6 | 1.253 | 0.085 | 1.209 | 0.699 | 0.427 | 0.409 | 1.738 | 1.054 |
| | 0 | 0 | 0 | 0 | 0 | 0 | 0 | 0 |
| APP | 6.896 | 5.065 | 9.373 | 7.186 | 10.913 | 9.454 | 16.049 | 15.715 |
| | 1 | 1 | 1 | 1 | 1 | 1 | 1 | 1 |
| GZMM | −0.326 | −0.517 | 0.132 | −0.759 | −0.587 | −0.25 | 0.009 | −0.928 |
| | 0 | 0 | 0 | 0 | 0 | 0 | 0 | 0 |
| USP2 | 5.18 | 3.153 | 3.123 | 2.396 | 0.914 | 1.541 | 4.618 | 3.803 |
| | 1 | 1 | 1 | 0 | 0 | 0 | 1 | 1 |
| ENSG00000117094 | 1.291 | −0.077 | 0.272 | −0.793 | 0.198 | −0.181 | 1.578 | 0.766 |
| | 0 | 0 | 0 | 0 | 0 | 0 | 0 | 0 |
| ADAMTS4 | −1.401 | −2.05 | −0.466 | −1.344 | 1.252 | 1.389 | 3.8 | 3.368 |
| | 0 | 1 | 0 | 0 | 0 | 0 | 1 | 1 |
| USP21 | −0.119 | 1.727 | | | 0.58 | 0.517 | | |
| | 0 | 0 | | | 0 | 0 | | |
| LTB4R | −1.058 | −0.402 | | | −0.399 | −0.393 | | |
| | 0 | 0 | | | 0 | 0 | | |

TABLE 4C

| | H22 | | | | H25 | | | |
|---|---|---|---|---|---|---|---|---|
| Screening | 1 μl | | | | 1 μl | | | |
| Infection | 3 | | | | 3 | | | |
| N for Act | DS | | PS | | DS | | PS | |
| cDNA | A | B | A | B | A | B | A | B |
| CDKN1A | 0.745 | 0.688 | 0.942 | 1.251 | 4.109 | 3.204 | 3.664 | 2.693 |
| | 0 | 0 | 0 | 0 | 1 | 1 | 1 | 0 |
| CSNK1G1 | 0.321 | 1.572 | −0.826 | −0.283 | 3.382 | 2.535 | 4.455 | 3.594 |
| | 0 | 0 | 0 | 0 | 1 | 0 | 1 | 1 |
| DGKE | 1.639 | 1.859 | −1.241 | −0.449 | 3.112 | 2.406 | 3.478 | 1.707 |
| | 0 | 0 | 0 | 0 | 1 | 0 | 1 | 0 |
| hRAS | 6.612 | 2.409 | 7.157 | 8.608 | 3.926 | 2.727 | 2.842 | 3.504 |
| | 1 | 0 | 1 | 1 | 1 | 0 | 0 | 1 |
| NR4A1 | −0.003 | 0.75 | −0.691 | 0.101 | 1.011 | 1.423 | 0.152 | 0.756 |
| | 0 | 0 | 0 | 0 | 0 | 0 | 0 | 0 |
| PREP | 0.779 | 1.562 | −0.517 | −0.433 | 3.554 | 2.623 | 4.121 | 4.455 |
| | 0 | 0 | 0 | 0 | 1 | 0 | 1 | 1 |
| PTPN6 | 1.701 | 1.2 | 1.778 | 1.854 | 4.409 | 3.371 | 4.052 | 2.828 |
| | 0 | 0 | 0 | 0 | 1 | 1 | 1 | 0 |
| SPINT1 | 4.007 | 1.396 | 2.169 | 2.344 | 4.196 | 3.282 | 1.866 | 2.209 |
| | 1 | 0 | 0 | 0 | 1 | 1 | 0 | 0 |
| SPC18 | 0.529 | −0.541 | 0.692 | 0.895 | 5.89 | 4.161 | 5.177 | 5.073 |
| | 0 | 0 | 0 | 0 | 1 | 1 | 1 | 1 |
| IMMP2L | −0.419 | 0.817 | 1.338 | 0.925 | 2.97 | 2.635 | 1.551 | 0.085 |
| | 0 | 0 | 0 | 0 | 0 | 0 | 0 | 0 |
| LOC166867 | 0.997 | 0.703 | 0.999 | 0.376 | 2.471 | 2.752 | 3.105 | 1.75 |
| | 0 | 0 | 0 | 0 | 0 | 0 | 1 | 0 |
| LOC148293 | 1.433 | 0.906 | 1.483 | 1.392 | 4.137 | 3.933 | 3.731 | 2.88 |
| | 0 | 0 | 0 | 0 | 1 | 1 | 1 | 0 |
| PSMA2 | 0.078 | −0.556 | 1.211 | 2.086 | 2.188 | 2.279 | 2.338 | 2.195 |
| | 0 | 0 | 0 | 0 | 0 | 0 | 0 | 0 |
| C14orf132 | 1.295 | 0.968 | −0.625 | −0.234 | 3.295 | 2.334 | 4.237 | 2.287 |
| | 0 | 0 | 0 | 0 | 1 | 0 | 1 | 0 |
| MAP3K8 | 0.893 | 3.729 | 0.228 | −0.006 | 0.949 | 0.851 | 0.147 | −0.55 |
| | 0 | 1 | 0 | 0 | 0 | 0 | 0 | 0 |
| NPUFA10 | 0.651 | 1.2 | 1.067 | 0.113 | 4.131 | 3.186 | 4.116 | 2.717 |
| | 0 | 0 | 0 | 0 | 1 | 1 | 1 | 0 |
| DAPK2 | 0.978 | 2.112 | −0.437 | −0.277 | 2.167 | 1.278 | 4.054 | 3.181 |
| | 0 | 0 | 0 | 0 | 0 | 0 | 1 | 1 |
| MAPK10 | 0.762 | 1.899 | −1.2 | −0.572 | 3.325 | 2.427 | 4.345 | 3.281 |
| | 0 | 0 | 0 | 0 | 1 | 0 | 1 | 1 |
| PDGFC | 4.195 | 1.399 | 3.549 | 2.683 | −0.524 | −0.406 | −0.381 | −0.143 |
| | 1 | 0 | 1 | 0 | 0 | 0 | 0 | 0 |
| NR1D2 | −1.9 | −0.707 | 0.779 | −0.612 | 1.064 | 3.277 | 1.599 | 2.383 |
| | 0 | 0 | 0 | 0 | 0 | 1 | 0 | 0 |
| | H25 | | | | H28 | | | |
| Screening | 0.20 μl | | | | 1 μl | | | |
| Infection | 3 | | | | 3 | | | |
| N for Act | DS | | PS | | DS | | PS | |
| cDNA | A | B | A | B | A | B | A | B |
| CDKN1A | 2.994 | 3.59 | 1.267 | 0.511 | −0568 | 0.007 | −0.009 | 1.455 |
| | 0 | 1 | 0 | 0 | 0 | 0 | 0 | 0 |
| CSNK1G1 | 1.866 | 2.476 | 1.102 | 2.401 | 1.794 | 2.483 | 2.294 | 2.232 |
| | 0 | 0 | 0 | 0 | 0 | 0 | 0 | 0 |
| DGKE | 1.563 | 1.682 | 1.178 | 2.33 | 2.695 | 2.063 | 0.778 | 1.483 |
| | 0 | 0 | 0 | 0 | 0 | 0 | 0 | 0 |
| hRAS | 5.632 | 5.814 | 4.743 | 2.714 | 7.952 | 4.047 | 8.338 | 8.311 |
| | 1 | 1 | 1 | 0 | 1 | 1 | 1 | 1 |
| NR4A1 | 3.278 | 3.747 | 1.959 | 3.16 | 0.328 | 0.04 | −0.615 | 0.212 |
| | 1 | 1 | 0 | 1 | 0 | 0 | 0 | 0 |
| PREP | 1.87 | 3.003 | 1.79 | 3.252 | 1.918 | 2.949 | 0.79 | 0.838 |
| | 0 | 1 | 0 | 1 | 0 | 0 | 0 | 0 |
| PTPN6 | 3.91 | 5.114 | 2.395 | 2.218 | −0.563 | 0.563 | 0.487 | 1.19 |
| | 1 | 1 | 0 | 0 | 0 | 0 | 0 | 0 |
| SPINT1 | 2.546 | 2.364 | 1.671 | 0.848 | 1.509 | 0.355 | 2.072 | 0.572 |
| | 0 | 0 | 0 | 0 | 0 | 0 | 0 | 0 |
| SPC18 | 5.84 | 6.34 | 3.589 | 3.743 | −1.75 | −1.288 | −1.033 | −0.371 |
| | 1 | 1 | 1 | 1 | 0 | 0 | 0 | 0 |
| IMMP2L | 3.267 | 3.827 | 1.604 | 4.08 | −1.128 | −0.77 | −0.165 | 0.185 |
| | 1 | 1 | 0 | 1 | 0 | 0 | 0 | 0 |
| LOC166867 | 3.798 | 4.32 | 1.493 | 1.359 | −0.916 | 0.09 | 0.086 | −0.433 |
| | 1 | 1 | 0 | 0 | 0 | 0 | 0 | 0 |
| LOC148293 | 4.191 | 5.176 | 2.125 | 1.941 | −0.236 | 0.394 | 0.603 | 1.027 |
| | 1 | 1 | 0 | 0 | 0 | 0 | 0 | 0 |
| PSMA2 | 3.593 | 3.935 | 1.642 | 1.633 | −1.335 | −1.115 | 0.2 | 0.782 |
| | 1 | 1 | 0 | 0 | 0 | 0 | 0 | 0 |
| C14orf132 | 1.65 | 1.771 | 2.037 | 2.897 | 2.804 | 1.991 | 0.789 | 2.421 |
| | 0 | 0 | 0 | 0 | 0 | 0 | 0 | 0 |
| MAP3K8 | 0.468 | 0.153 | 1.258 | 1.049 | 2.39 | 4.373 | 4.67 | 3.948 |
| | 0 | 0 | 0 | 0 | 0 | 1 | 1 | 1 |
| NPUFA10 | 1.126 | 2.041 | 2.54 | 3.035 | 0.939 | 1.204 | 0.835 | 0.031 |
| | 0 | 0 | 0 | 1 | 0 | 0 | 0 | 0 |
| DAPK2 | 0.972 | 1.959 | 0.765 | 1.7 | 2.208 | 4.197 | 2.578 | 3.677 |
| | 0 | 0 | 0 | 0 | 0 | 1 | 0 | 1 |
| MAPK10 | 1.606 | 2.086 | 1.281 | 2.667 | 2.271 | 2.804 | 0.897 | 1.36 |
| | 0 | 0 | 0 | 0 | 0 | 0 | 0 | 0 |
| PDGFC | −0.254 | −0.216 | −0.13 | −0.768 | 2.68 | 1.696 | 2.405 | 0.321 |
| | 0 | 0 | 0 | 0 | 0 | 0 | 0 | 0 |
| NR1D2 | 0.475 | 1.826 | 3.81 | 4.256 | −1.461 | −0.521 | −1.408 | −1.875 |
| | 0 | 0 | 1 | 1 | 0 | 0 | 0 | 0 |

TABLE 4D

| | H22 | | | | H25 | | | |
|---|---|---|---|---|---|---|---|---|
| Screening | 1 μl | | | | 1 μl | | | |
| Infection | 2 | | | | 1.7 | | | |
| N for Rep | DS | | PS | | DS | | PS | |
| cDNA | A | B | A | B | A | B | A | B |
| HTR2B | 1.834 | 1.621 | 2.767 | 1.436 | −1.961 | −1.72 | −1.407 | −1.273 |
| | 0 | 0 | 0 | 0 | 1 | 1 | 0 | 0 |
| MARK1 | −1.479 | 0.173 | −0.429 | −0.688 | −1.76 | −1.794 | −1.674 | −1.641 |
| | 0 | 0 | 0 | 0 | 1 | 1 | 0 | 0 |
| PIP5K1A | −1.517 | −0.59 | −1.113 | −0.974 | −1.473 | −1.104 | −1.721 | −1.978 |
| | 0 | 0 | 0 | 0 | 0 | 0 | 1 | 1 |
| | H25 | | | | H28 | | | |
| Screening | 0.25 μl | | | | 1 μl | | | |
| Infection | 2 | | | | 3 | | | |
| N for Rep | DS | | PS | | DS | | PS | |
| cDNA | A | B | A | B | A | B | A | B |
| HTR2B | −0.838 | −0.848 | −0.733 | −0.8 | 3.959 | 4.254 | 1.821 | 1.523 |
| | 0 | 0 | 0 | 0 | 1 | 1 | 0 | 0 |
| MARK1 | −1.891 | −2.024 | −1.684 | −1.51 | −1.205 | 0.496 | 0.911 | 0.314 |
| | 0 | 0 | 0 | 0 | 0 | 0 | 0 | 0 |
| PIP5K1A | −0.504 | −1.216 | −0.996 | −1.114 | −1.426 | −1.209 | −1.33 | −1.733 |
| | 0 | 0 | 0 | 0 | 0 | 0 | 0 | 0 |
| | H22 | | | | H25 | | | |
| Screening | 1 μl | | | | 1 μl | | | |
| Infection | 2 | | | | 2 | | | |
| N for Rep | DS | | PS | | DS | | PS | |
| cDNA | A | B | A | B | A | B | A | B |
| FLJ23516 | −2.339 | −2.611 | −2.091 | −2.397 | −2.348 | −2.449 | −2.114 | −2.15 |
| | 0 | 1 | 1 | 1 | 1 | 1 | 1 | 1 |
| | H25 | | | | H28 | | | |
| Screening | 0.25 μl | | | | 1 μl | | | |
| Infection | 1.5 | | | | 2.5 | | | |
| N for Rep | DS | | PS | | DS | | PS | |
| cDNA | A | B | A | B | A | B | A | B |
| FLJ23516 | −2.421 | −2.545 | −1.803 | −1.697 | −2.571 | −3.09 | −1.51 | −1.376 |
| | 1 | 1 | 1 | 1 | 1 | 1 | 0 | 0 |

The experimental work following this initial screening indicates that the LIGASE identified as RNF128 (SEQ ID NO: 1 (DNA sequence) and 2 (amino acid sequence)) are involved in APP processing.

Following the initial screening work, additional screening of the LIGASE in Hek293 APPwt cells demonstrated that increased expression thereof lead to (a) decreased levels of amyloid peptide 1-42. These results indicate that induction of RNF128 inhibits the production and processing of amyloid beta peptides.

The DNA and amino acid sequence information for RNF128 is provided in Table 1, while the sequence information for the protein domains of RNF128 are described in Table 5 below.

TABLE 5

| SEQ | | | | |
|---|---|---|---|---|
| ID | | Protein | | |
| No. | Accession | Segment | Sequence | Name |
| 235 | NM_194 | Extracellular | MGPPPGAGVSCRGGCGFSRLL | RNF128 |
| | 463 | Domain | AWCFLLALSPQAPGSRGAEAV | |
| | | | WTAYLNVSWRVPHTGVNRTV | |
| | | | WELSEEGVYGQDSPLEPVAGV | |
| | | | LVPPDGPGALNACNPHTNFTV | |
| | | | PTVWGSTVQVSWLALIQRGG | |
| | | | GCTFADKIHLAYERGASGAVI | |
| | | | FNFPGTRNEVIPMSHPGAVDIV | |
| | | | AIMIGNLKGTKILQSIQRGIQV | |
| | | | TMVIEVGKKHGPWVNH | |
| 236 | NM_194 | Intracellular | YSARRLRNARAQSRKQRQLK | RNF128 |
| | 463 | Domain | ADAKKAIGRLQLRTLKQGDK | |
| | | | EIGPDGDSCAVCIELYKPNDLV | |
| | | | RILTCNHIFHKTCVDPWLLEH | |
| | | | RTCPMCKCDILKALGIEVDVE | |
| | | | DGSVSLQVPVSNEISNSASSHE | |
| | | | EDNRSETASSGYASVQGTDEP | |
| | | | PLEEHVQSTNESLQLVNHEAN | |
| | | | SVAVDVIPHVDNPTFEEDETP | |
| | | | NQETAVREIKS | |
| 237 | NM_194 | Transmembrane | YSIFFVSVSFFIITAATVGYFIF | RNFI28 |
| | 463 | Domain | | |

### EXAMPLE 2

### Expression of LIGASE in the Human Brain

Upon identification of a modulator of APP processing, it is important to evaluate whether the modulator is expressed in the tissue and the cells of interest. This can be achieved by measuring the RNA and/or protein levels in the tissue and cells. In recent years, RNA levels are being quantified through real time PCR technologies, whereby the RNA is first transcribed to cDNA and then the amplification of the cDNA of interest is monitored during a PCR reaction. The amplification plot and the resulting Ct value are indicators for the amount of RNA present in the sample. Ct values are determined in the presence or absence of the reverse transcriptase step (+RT versus −RT). An amplification signal in the −RT condition indicates the occurrence of non-specific PCR products originating from the genomic DNA. If the +RT Ct value is 3 Ct values higher than the −RT Ct value, then the investigated RNA is present in the sample.

To assess whether the identified LIGASE is expressed in the human brain, real time PCR with specific primers for the LIGASE is performed on human total brain, human cerebral cortex, and human hippocampal total RNA (BD Biosciences)(see Table 6). In addition, to assess the neuronal expression, the expression analysis was also performed on RNA samples prepared from mouse or rat primary neuron cell cultures using PCR primers for the murine or rat homolog of the polypeptide of the invention.

TABLE 6

Primers used in the quantitative real time
PCR analysis for LIGASE-expression

| | | | | SEQ ID |
|---|---|---|---|---|
| Gene | Species | Primer name | Sequence | NO. |
| RNF128 | H. Sapiens | RNF128_Hs_For | CGCATCATCTGGATATGCTTCA | 6 |
| | H. Sapiens | RNF128_Hs_Rev | ACCAGCTGTAGACTTTCATTTGT | 7 |
| | | | TGAG | |
| RNF128 | M. Musculus | RNF128_Mm_For | TCTGGGAATTGAGGTGGATGT | 8 |
| | M. Musculus | RNF128_Mm_Rev | CACTGCGACTGTCCTCTTCATG | 9 |

Forty ng of RNA are reverse-transcribed to DNA using the MultiScribe Reverse Transcriptase (50 U/μl) enzyme (Applied BioSystems). The resulting cDNA is amplified with AmpliTaq Gold DNA polymerase (Applied BioSystems) during 40 cycles using an ABI PRISMS 7000 Sequence Detection System. Amplification of the transcript is detected via SybrGreen which results in a fluorescent signal upon intercalation in double stranded DNA.

Total RNA isolated from mouse primary neurons and human total brain, cerebral cortex and hippocampal are analyzed for the presence of the LIGASE transcripts via quantitative real time PCR. The Ct values for RNF128 indicate that they are detected in all RNA samples (Table 7).

To gain more insight into the specific cellular expression, immunohistochemistry (protein level) and/or in situ hybridization (RNA level) are carried out on sections from human normal and Alzheimer's brain hippocampal, cortical and subcortical structures. These results indicate whether expression occurs in neurons, microglia cells, or astrocytes. The comparison of diseased tissue with healthy tissue indicates whether the LIGASE is expressed in the diseased tissue and whether its expression level is changed compared to the non-pathological situation.

TABLE 7

| Total RNA isolated from human brain, human | | Ct values | |
|---|---|---|---|
| cerebral cortex, human hippocampus, and | | obtained during | |
| mouse or rat primary hippocampal neuronal | | quantitative | |
| cultures, is tested for the presence of the | | real time PCR | |
| respective RNA via quantitative realtime PCR. | | Ct | |
| Gene | Tissue | RT+ | RT− |
| RNF128 | Human Brain | 24, 35 | 40 |
| | Human Brain Hippocampus | 24, 94 | 39, 35 |
| | Human Brain Cerebral Cortex | 24, 32 | 40 |
| RNF128 | Mus Musculus Primary Neurons | 24, 64 | 30, 63 |

### EXAMPLE 3

### Amyloid Beta Peptide Reduction Via LIGASE Over-expression

The inhibitory effect of RNF128 is confirmed upon re-screening of the viruses with a known titer (viral particles/ml), as determined by quantitative real time PCR. RNF128 virus is infected at MOIs ranging from 2 to 1250 and the experiment is performed as described above. In addition, the effect of RNF128 on amyloid beta 1-42 and amyloid beta x-42, amyloid beta 1-40, and amyloid beta 1-y levels, is checked under similar conditions as above. The respective ELISAs are performed as described above, except that the following antibodies were used: for the amyloid beta 1-40 ELISA, the capture and detection antibody are respectively JRF/cAbeta40/10 and JRF/AbetaN/25-HRP (obtained from M Mercken, Johnson and Johnson Pharmaceutical Research and Development, B-2340 Beerse, Belgium), for the amyloid beta 11-42 ELISA, the capture and detection antibody are respectively JRF/cAbeta42/26 and JRF/hAb11/1 (obtained from M Mercken, Johnson and Johnson Pharmaceutical Research and Development, B-2340 Beerse, Belgium), for the amyloid beta x-42 ELISA (x ranges from 1-17), the capture and detection antibody are respectively JRF/cAbeta42/26 and 4G8-HRP (obtained respectively from M Mercken, Johnson and Johnson Pharmaceutical Research and Development, B-2340 Beerse, Belgium and from Signet, USA) while for the amyloid beta 1-y ELISA (y ranges from 24-42) the capture and detection antibodies are JRF/AbetaN/25 and 4G8-HRP, respectively (obtained respectively from M Mercken, Johnson and Johnson Pharmaceutical Research and Development, B-2340 Beerse, Belgium and from Signet, USA). The amyloid beta 1-y ELISA is used for the detection of amyloid peptides with a variable C-terminus (amyloid beta 1-37; 1-38; 1-39; 1-40; 1-42).

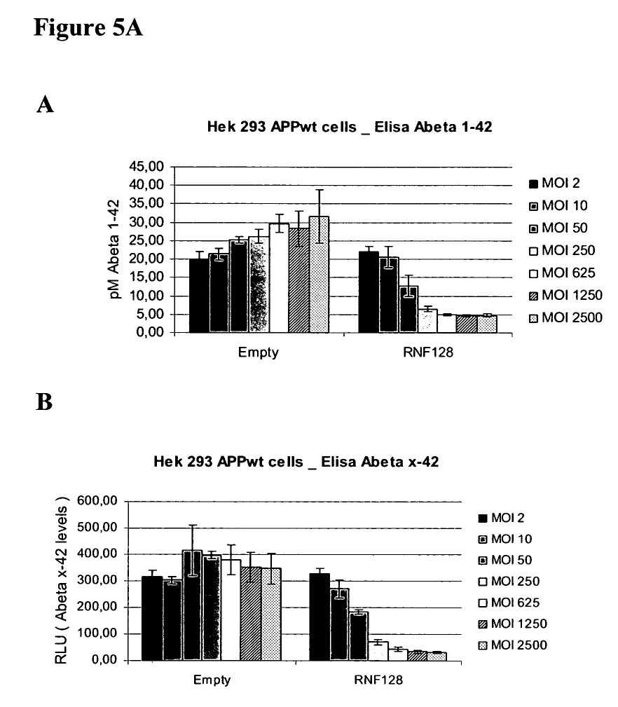

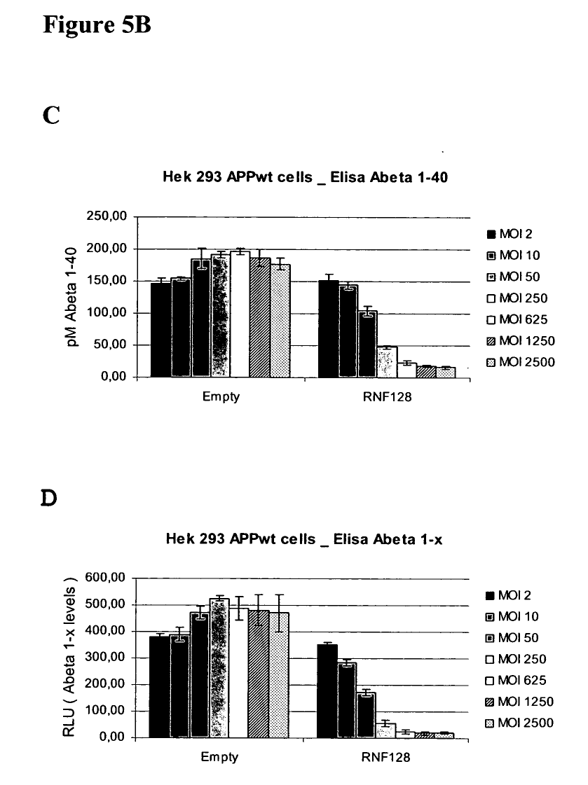

The results are presented in FIGS. 5A-5B. Expression inhibits all types of amyloid beta peptide measured in a dose-dependant manner.

### EXAMPLE 4

### Amyloid Beta Production in Rat Primary Neuronal Cells

To investigate whether RNF128 affects amyloid beta production in a primary neuron, human or rat primary hippocampal or cortical neurons are transduced with adenovirus containing the RNF128 cDNA. Amyloid beta levels are determined by ELISA (see EXAMPLE 1). Since rodent APP genes carry a number of mutations in APP compared to the human sequence, they produce less amyloid beta 1-40 and 1-42. To achieve higher amyloid beta levels, the neurons are co-transduced with adenovirus containing cDNA for RNF128 and with cDNA coding for human wild type APP or human Swedish mutant APP (which enhances amyloid beta production).

Rat primary neuron cultures are prepared from brain of E18-E19-day-old fetal Sprague Dawley rats according to Goslin and Banker (Culturing Nerve cells, second edition, 1998 ISBN 0-262-02438-1). Single cell suspensions obtained from the hippocampus or cortices are prepared. The number of viable cells is determined and plated on poly-L-lysine-coated plastic 96-well plates in minimal essential medium (MEM) supplemented with 10% horse serum. The cells are seeded at a density of 50,000 cells per well (i.e. about 166,000 cells/cm²). After 3-4 h, culture medium is replaced by 160-μl serum-free neurobasal medium with B27 supplement (GIBCO BRL). Cytosine arabinoside (5 μM) is added 24 h after plating to prevent non-neuronal (glial) cell proliferation.

Neurons are used at day 5 after plating. Before adenoviral transduction, 150 μl-conditioned medium of these cultures is transferred to the corresponding wells in an empty 96-well plate and 50 μl of the conditioned medium is returned to the cells. The remaining 100 μl/well is stored at 37° C. and 5% CO₂. Hippocampal primary neuron cultures are infected with the crude lysate of Ad5C09Att00/A011200-RNF128, Ad5C09Att00/A010801-LacZ_v1, Ad5C09Att00/A010800-eGFP_v1 and Ad5C09Att00/A010800-luc_v17 viruses containing the cDNA of RNF128, LacZ, eGFP and luciferase respectively at different MOIs, ranging from 250 to 2000. In addition the cells are co-infected with the purified adenovirus Ad5C01Att01/A010800 APP_v6 expressing human wild type APP695 at an MOI of 2000. Sixteen to twenty-four hours after transduction, virus is removed and cultures are washed with 100-μl pre-warmed fresh neurobasal medium. After removal of the wash solution, new medium, containing 50 μl of the stored conditioned medium and 50 μl of fresh neurobasal medium, is transferred to the corresponding cells. Medium is harvested after 48 and 72 hours. The cell number in the wells is determined by assessing the ATP levels. Amyloid beta concentration is determined by amyloid beta 1-42 specific ELISA (see EXAMPLE 1). Amyloid beta 1-42 levels are normalized for cell number.

### EXAMPLE 5

### Ligand Screens for LIGASE

Screening for Compounds that Bind to the Ligase Polypeptides (Displacement Experiment)

Compounds are screened for binding to the RNF128 polypeptide. The affinity of the compounds to the polypeptide is determined in a displacement experiment. In brief, the LIGASE polypeptide is incubated with a labeled (radiolabeled, fluorescent labeled) ligand that is known to bind to the polypeptide and with an unlabeled compound. The displacement of the labeled ligand from the polypeptide is determined by measuring the amount of labeled ligand that is still associated with the polypeptide. The amount associated with the polypeptide is plotted against the concentration of the compound to calculate IC₅₀ values. This value reflects the binding affinity of the compound to its target, i.e. the RNF128 polypeptide. Strong binders have an IC₅₀ in the nanomolar and even picomolar range. Compounds that have an IC₅₀ of at least 10 micromol or better (nmol to pmol) are applied in beta amyloid secretion assay to check for their effect on the beta amyloid secretion and processing. The RNF128 polypeptide can be prepared in a number of ways depending on whether the assay is run on cells, cell fractions or biochemically, on purified proteins.

Ligand Binding Study On Cell Surface

RNF128 is expressed in mammalian cells (Hek293, CHO, COS7) by adenoviral transducing the cells (see U.S. Pat. No. 6,340,595). The cells are incubated with both labeled ligand (iodinated, tritiated, or fluorescent) and the unlabeled compound at various concentrations, ranging from 10 pM to 10 μM (3 hours at 4° C.: 25 mM HEPES, 140 mM NaCl, 1 mM CaCl₂, 5 mM MgCl₂ and 0.2% BSA, adjusted to pH 7.4). Reactions mixtures are aspirated onto PEI-treated GF/B glass filters using a cell harvester (Packard). The filters are washed twice with ice cold wash buffer (25 mM HEPES, 500 mM NaCl, 1 mM CaCl₂, 5 mM MgCl₂, adjusted to pH 7.4). Scintillant (MicroScint-10; 35 μl) is added to dried filters and the filters counted in a (Packard Topcount) scintillation counter. Data are analyzed and plotted using Prism software (GraphPad Software, San Diego, Calif.). Competition curves are analyzed and IC₅₀ values calculated. If one or more data points do not fall within the sigmoidal range of the competition curve or close to the sigmoidal range the assay is repeated and concentrations of labeled ligand and unlabeled compound adapted to have more data points close to or in the sigmoidal range of the curve.

Ligand Binding Studies on Membrane Preparations

Membrane preparations are isolated from mammalian cells (Hek293, CHO, COS7) over-expressing the LIGASE as follows: Medium is aspirated from the transduced cells and cells are harvested in 1×PBS by gentle scraping. Cells are pelleted (2500 rpm 5 min) and resuspended in 50 mM Tris pH 7.4 (10×10⁶ cells/ml). The cell pellet is homogenized by sonicating 3×5 sec (UP50H; sonotrode MS1; max amplitude: 140 μm; max Sonic Power Density: 125W/cm²). Membrane fractions are prepared by centrifuging 20 min at maximal speed (13000 rpm˜15 000 to 20 000 g or rcf). The resulting pellet is resuspended in 500 μl 50 mM Tris pH 7.4 and sonicated again for 3×5 sec. The membrane fraction is isolated by centrifugation and finally resuspended in PBS. Binding competition and derivation of IC₅₀ values are determined as described above.

1. A method for identifying a compound that inhibits the processing of amyloid-beta precursor protein in a mammalian cell, comprising
(a) contacting a compound with a polypeptide comprising an amino acid sequence selected from the group consisting of SEQ ID NO: 2 and 3-5; and
(b) measuring a compound-polypeptide property related to the production of amyloid-beta protein.

2. The method according to claim 1, wherein said polypeptide comprises SEQ ID NO: 3-5 in an in vitro cell-free preparation.

3. The method according to claim 1, wherein said polypeptide is membrane-bound.

4. The method according to claim 2, wherein said polypeptide is present as a transmembrane protein in a mammalian cell.

5. The method of claim 1, wherein said property is a binding affinity of said compound to said polypeptide.

6. The method of claim 4, wherein said property is activation of a biological pathway producing an indicator of the processing of amyloid-beta precursor protein.

7. The method of claim 6 wherein said indicator is amyloid-beta peptide.

8. The method of claim 7 wherein said amyloid-beta protein is selected from the group consisting of one or more of amyloid-beta peptide 1-42, 1-40, 11-42 and 11-40.

9. The method of claim 8 wherein said amyloid-beta protein is amyloid-beta peptide 1-42.

10. The method of claim 9 wherein said amyloid-beta protein is amyloid-beta peptide x-42 and/or 1-y.

12. The method according to claim 1, wherein said compound is selected from the group consisting of compounds of a commercially available screening library and compounds that have been demonstrated to have binding affinity for a polypeptide comprising an amino acid sequence selected from the group consisting of SEQ ID NO: 2 and 3-5.

13. The method according to claim 12, wherein said compound is a peptide in a phage display library or an antibody fragment library.

14. A pharmaceutical composition for the treatment or prevention of a condition involving cognitive impairment or a susceptibility to the condition, comprising a nucleic acid sequence encoding a polypeptide comprising an amino acid sequence selected from the group consisting of SEQ ID NO: 2 and 3-5, in admixture with a pharmaceutically acceptable carrier.

15. The pharmaceutical composition according to claim 14, wherein a vector in a mammalian cell expresses said nucleic acid sequence.

16. The pharmaceutical composition according to claim 15, wherein said vector is an adenoviral, retroviral, adeno-associated viral, lentiviral, a herpes simplex viral or a sendaiviral vector.

17. The pharmaceutical composition according to claim 16, wherein said nucleic acid sequence is SEQ ID NO: 1.

18. A method of inhibiting the aberrant processing of amyloid-beta precursor protein in a subject suffering or susceptible to the abnormal processing of said protein, comprising administering to said subject a pharmaceutical composition according to claim 14.

19. A method according to claim 18 for treatment or prevention of a condition involving cognitive impairment or a susceptibility to the condition.

20. The method according to claim 19 wherein the condition is Alzheimer's disease.

21. A pharmaceutical composition for the treatment or prevention of a condition involving cognitive impairment or a susceptibility to the condition, comprising an effective amyloid-beta peptide x-42-inducing amount of vector comprising a nucleic acid sequence of SEQ ID NO: 1 in admixture with a pharmaceutically acceptable carrier.


SEQUENCE LISTING (9 sequences)

SEQ ID NO: 1 - DNA, 2851 bp, Homo sapiens
gcttcacgctaaagccccagagcccgacgcggcagccgcggtagcggagaagactggagctccgaggagctgcatctgcggcaacctgtgtgctgacgctacgtgcctcctggctccgacgtagctcgcagctccccagtctcactccattccttccccacctggcgcgcacctgctcaagaccagggtcctgccaagcgctaggagggcgcgtgccaggggcgctagggaactgcggagcgcgcgcgccatggggccgccgcctggggccggggtctcctgccgcggtggctgcggcttttccagattgctggcatggtgcttcctgctggccctgagtccgcaggcacccggttcccggggggctgaagcagtgtggaccgcgtacctcaacgtgtcctggcgggttccgcacacgggagtgaaccgtacggtgtgggagctgagcgaggagggcgtgtacggccaggactcgccgctggagcctgtggctggggtcctggtaccgcccgacgggcccggggcgcttaacgcctgtaacccgcacacgaatttcacggtgcccacggtttggggaagcaccgtgcaagtctcttggttggccctcatccaacgcggcgggggctgcaccttcgcagacaagatccatctggcttatgagagaggggcgtctggagccgtcatctttaacttccccgggacccgcaatgaggtcatccccatgtctcacccgggtgcagtagacattgttgcaatcatgatcggcaatctgaaaggcacaaaaattctgcaatctattcaaagaggcatacaagtgacaatggtcatagaagtagggaaaaaacatggcccttgggtgaatcactattcaatttttttcgtttctgtgtccttttttattattacggcggcaactgtgggctattttatcttttattctgctcgaaggctacggaatgcaagagctcaaagcaggaagcagaggcaattaaaggcagatgctaaaaaagctattggaaggcttcaactacgcacactgaaacaaggagacaaggaaattggccctgatggagatagttgtgctgtgtgcattgaattgtataaaccaaatgatttggtacgcatcttaacgtgcaaccatattttccataagacatgtgttgacccatggctgttagaacacaggacttgccccatgtgcaaatgtgacatactcaaagctttgggaattgaggtggatgttgaagatggatcagtgtctttacaagtccctgtatccaatgaaatatctaatagtgcctcctcccatgaagaggataatcgcagcgagaccgcatcatctggatatgcttcagtacagggaacagatgaaccgcctctggaggaacacgtgcagtcaacaaatgaaagtctacagctggtaaaccatgaagcaaattctgtggcagtggatgttattcctcatgttgacaacccaacctttgaagaagacgaaactcctaatcaagagactgctgttcgagaaattaaatcttaaaatctgtgtaaatagaaaacttgaaccattagtaataacagaactgccaatcagggcctagtttctattaataaattggataaatttaataaaataagagtgatactgaaagtgctcagatgactaatattatgctatagttaaatggcttaaaatatttaacctgttaacttttttccacaaactcattataatatttttcataggcaagtttcctctcagtagtgataacaacatttttagacattcaaaactgtcttcaagaagtcacgtttttcatttataacaattttcttataaaaacatgttgcttttaaaatgtggagtagctgtaatcactttattttatgatagtatcttaatgaaaaatactacttctttagcttgggctacatgtgtcagggtttttctccaggtgcttatattgatctggaattgtaatgtaaaaagcaatgcaaacttaggcgagtacttcttgaaatgtctatttaagctgctttaagttaatagaaaagattaaagcaaaatattcatttttactttttcttatttttaaaattaggctgaatgtacttcatgtgatttgtcaaccatagtttatcagagattatggacttaattgattggtatattagtgacatcaacttgacacaagattagacaaaaaattccttacaaaaatactgtgtaactatttctcaaacttgtgggatttttcaaaagctcagtatatgaatcatcatactgtttgaaattgctaatgacagagtaagtaacactaatattggtcattgatcttcgttcatgaattagtctacagaaaaaaaatgttctgtaaaattagtctgttgaaaatgttttccaaacaatgttactttgaaaattgagtttatgtttgacctaaatgggctaaaattacattagataaactaaaattctgtccgtgtaactataaattttgtgaatgcattttcctggtgtttgaaaaagaagggggggagaattccaggtgccttaatataaagtttgaagcttcatccaccaaagttaaatagagctatttaaaaatgcactttatttgtactctgtgtggcttttgttttagaattttgttcaaattatagcagaatttaggcaaaaataaaacagacatgtatttttgtttgctgaatggatgaaaccattgcattcttgtacactgatttgaaatgctgtaaatatgtcccaatttgtattgattctctttaaatataaaatgtaaataaaatattccaataaaagtttgtgtctggtgttagtttaaaaaaaaaaaaa

SEQ ID NO: 2 - protein, 428 aa, Homo sapiens
MGPPPGAGVSCRGGCGFSRLLAWCFLLALSPQAPGSRGAEAVWTAYLNVSWRVPHTGVNRTVWELSEEGVYGQDSPLEPVAGVLVPPDGPGALNACNPHTNFTVPTVWGSTVQVSWLALIQRGGGCTFADKIHLAYERGASGAVIFNFPGTRNEVIPMSHPGAVDIVAIMIGNLKGTKILQSIQRGIQVTMVIEVGKKHGPWVNHYSIFFVSVSFFIITAATVGYFIFYSARRLRNARAQSRKQRQLKADAKKAIGRLQLRTLKQGDKEIGPDGDSCAVCIELYKPNDLVRILTCNHIFHKTCVDPWLLEHRTCPMCKCDILKALGIEVDVEDGSVSLQVPVSNEISNSASSHEEDNRSETASSGYASVQGTDEPPLEEHVQSTNESLQLVNHEANSVAVDVIPHVDNPTFEEDETPNQETAVREIKS

SEQ ID NO: 3 - protein, 205 aa, Artificial Sequence
  RNF128 domain
MGPPPGAGVSCRGGCGFSRLLAWCFLLALSPQAPGSRGAEAVWTAYLNVSWRVPHTGVNRTVWELSEEGVYGQDSPLEPVAGVLVPPDGPGALNACNPHTNFTVPTVWGSTVQVSWLALIQRGGGCTFADKIHLAYERGASGAVIFNFPGTRNEVIPMSHPGAVDIVAIMIGNLKGTKILQSIQRGIQVTMVIEVGKKHGPWVNH

SEQ ID NO: 4 - protein, 200 aa, Artificial Sequence
  RNF128 domain
YSARRLRNARAQSRKQRQLKADAKKAIGRLQLRTLKQGDKEIGPDGDSCAVCIELYKPNDLVRILTCNHIFHKTCVDPWLLEHRTCPMCKCDILKALGIEVDVEDGSVSLQVPVSNEISNSASSHEEDNRSETASSGYASVQGTDEPPLEEHVQSTNESLQLVNHEANSVAVDVIPHVDNPTFEEDETPNQETAVREIKS

SEQ ID NO: 5 - protein, 23 aa, Artificial Sequence
  RNF128 domain
YSIFFVSVSFFIITAATVGYFIF

SEQ ID NO: 6 - DNA, 22 bp, Artificial Sequence
  PCR primer
cgcatcatctggatatgcttca

SEQ ID NO: 7 - DNA, 27 bp, Artificial Sequence
  PCR primer
accagctgtagactttcatttgttgac

SEQ ID NO: 8 - DNA, 21 bp, Artificial Sequence
  PCR primer
tctgggaattgaggtggatgt

SEQ ID NO: 9 - DNA, 22 bp, Artificial Sequence
  PCR primer
cactgcgactgtcctcttcatg